(19) United States
(12) Patent Application Publication
(10) Pub. No.: US 2024/0409981 A1
(43) Pub. Date: Dec. 12, 2024

(54) CHARACTERIZING ANALYTES IN A SAMPLE USING NORMALIZED SIGNALS

(71) Applicant: Illumina, Inc., San Diego, CA (US)

(72) Inventors: Anurag Agrawal, San Diego, CA (US); Jeffrey Brodin, San Diego, CA (US); Jeffrey Fisher, San Diego, CA (US); Bo Lu, San Diego, CA (US); Chenlu Pan, San Diego, CA (US)

(73) Assignee: Illumina, Inc., San Diego, CA (US)

(21) Appl. No.: 18/569,421

(22) PCT Filed: Jul. 21, 2022
(86) PCT No.: PCT/US22/37914

§ 371 Date: Dec. 12, 2023

Related U.S. Application Data
(60) Provisional application No. 63/225,035, filed on Jul. 23, 2021.

(51) Int. Cl. C12Q 1/6818; C12Q 1/6827

(52) U.S. Cl. CPC C12Q 1/6818; C12Q 1/6827

(57) ABSTRACT

Some examples relate to a method for characterizing polynucleotides in a sample. First polynucleotides coupled to a first substrate may be hybridized to second polynucleotides in a sample. First labeled nucleotides may be added to the first polynucleotides using a sequence of the second polynucleotides. A first signal intensity from the first labeled nucleotides is measured. Second labeled nucleotides are added to the first polynucleotides using the sequence of the second polynucleotides. A second signal intensity from the second labeled nucleotides is measured. The first signal intensity is normalized using the second signal intensity. The normalized first signal intensity characterizes the second polynucleotides in the sample.

### CROSS-REFERENCE TO RELATED APPLICATION

This application claims the benefit of U.S. Provisional Patent Application No. 63/225,035, filed on Jul. 23, 2021 and entitled “CHARACTERIZING ANALYTES IN A SAMPLE USING NORMALIZED SIGNALS”, the entire contents of which are incorporated by reference herein.

### FIELD

This application relates to methods of characterizing polynucleotides using normalized signals.

### BACKGROUND

The detection of specific nucleotide sequences present in a biological sample has been used, for example, as a method for identifying and classifying microorganisms, diagnosing infectious diseases, detecting and characterizing genetic abnormalities, identifying genetic changes associated with cancer, studying genetic susceptibility to diseases, and measuring response to various types of treatment. A common technique for detecting specific nucleotide sequences in a biological sample is polynucleotide sequencing.

Polynucleotide sequencing methodology has evolved from the chemical degradation methods used by Maxam and Gilbert and the strand elongation methods used by Sanger. Several sequencing methodologies are now in use which allow for the parallel processing of thousands of polynucleotides all on a single chip. Some platforms include bead-based and microarray formats in which silica beads are functionalized with probes depending on the application of such formats in applications including sequencing, genotyping, or gene expression profiling.

Some sequencing systems use fluorescence-based detection, whether for “sequencing-by-synthesis” or for genotyping, in which a given nucleotide is labeled with a fluorescent label, and the nucleotide is identified based on detecting the fluorescence from that label.

### SUMMARY

The present application relates to characterizing analytes in a sample using normalized signals.

Some examples provide a method for characterizing polynucleotides in a sample. The method may include hybridizing first polynucleotides coupled to a first substrate to second polynucleotides in a sample. The method may include adding first labeled nucleotides to the first polynucleotides using a sequence of the second polynucleotides. The method may include measuring a first signal intensity from the first labeled nucleotides. The method may include adding second labeled nucleotides to the first polynucleotides using the sequence of the second polynucleotides. The method may include measuring a second signal intensity from the second labeled nucleotides. The method may include normalizing the first signal intensity using the second signal intensity. The normalized first signal intensity may characterize the second polynucleotides in the sample.

In some examples, the second labeled nucleotides are added after the first labeled nucleotides are added. In some examples, the second labeled nucleotides are added before the first labeled nucleotides are added.

In some examples, the method further includes hybridizing third polynucleotides coupled to a second substrate to fourth polynucleotides in the sample. The method may include adding third labeled nucleotides to the third polynucleotides using a sequence of the fourth polynucleotides. The method may include measuring a third signal intensity from the third labeled nucleotides. The method may include adding fourth labeled nucleotides to the third polynucleotides using the sequence of the fourth polynucleotides. The method may include measuring a fourth signal intensity from the fourth labeled nucleotides. The method may include normalizing the third signal intensity using the fourth signal intensity. The normalized third signal intensity may characterize the fourth polynucleotides in the sample.

Some examples further include calculating a difference or ratio between an amount of the second polynucleotides in the sample and an amount of the fourth polynucleotides in the sample using a difference or ratio between the normalized first signal intensity and the normalized third signal intensity. In some examples, the fourth polynucleotides have a different sequence than the second polynucleotides. In some examples, the first substrate includes a first bead, and wherein the second substrate includes a second bead. In some examples, the first bead is located within a first well, and the second bead is located within a second well.

In some examples, the normalizing corrects for a difference between the first signal intensity and the third signal intensity that is caused by a position of the first bead within the first well or a position of the second bead within the second well. In some examples, the normalizing corrects for a difference between the first signal intensity and the third signal intensity that is caused by surface characteristic of the first well or a surface characteristic of the second well. In some examples, the normalizing corrects for a difference between the first signal intensity and the third signal intensity that is caused by a loading condition of the first bead within the first well or a loading condition of the second bead within the second well. In some examples, the normalizing corrects for a difference between the first signal intensity and the third signal intensity that is caused by a capture efficiency of the first bead within the first well or a capture efficiency of the second bead within the second well.

In some examples, the normalizing corrects for a difference between the first signal intensity and the third signal intensity that is caused by a size of the first bead or a size of the second bead.

In some examples, the normalizing corrects for a difference between the first signal intensity and the third signal intensity that is caused by a number of the second polynucleotides coupled to the first substrate or a number of the fourth polynucleotides coupled to the second substrate.

In some examples, the normalized first signal intensity corresponds to an amount of a first single nucleotide polymorphism (SNP) in the sample, and the normalized third signal intensity corresponds to an amount of a second, different SNP in the sample. In some examples, the normalized first signal intensity corresponds to an amount by which a first base is methylated in the sample, and the normalized third signal intensity corresponds to an amount by which the first base is not methylated in the sample.

In some examples, the first and second polynucleotides are hybridized to one another in solution.

In some examples, the first substrate includes a bead. In some examples, the method further includes capturing the bead within a well. In some examples, the first and second labeled nucleotides are added after the bead is captured in the well. In some examples, the first and second signal intensities are measured using a complementary metal oxide semiconductor (CMOS) sensor on which the well is disposed.

In some examples, the second labeled nucleotide is added to a position adjacent to where the first labeled nucleotide is added.

In some examples, the method further includes adding one or more additional nucleotides to positions between where the first and second labeled nucleotides respectively are added.

In some examples, the first and second labeled nucleotides respectively include first and second fluorophores.

In some examples, characterizing the second polynucleotides in the sample includes determining an amount of the second polynucleotides in the sample, identifying the first nucleotide, or both determining an amount of the second polynucleotides in the sample and identifying the first nucleotide.

In some examples, the second labeled nucleotide is coupled to a primer hybridized to a barcode oligonucleotide coupled to the first substrate.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1E schematically illustrate example compositions and operations used to characterize analytes in a sample using normalized signals.

FIGS. 2A-2C schematically illustrate example raw signal intensities that may be obtained using the compositions and operations described with reference to FIG. 1C.

FIGS. 3A-3C schematically illustrate example raw signal intensities that may be obtained using the compositions and operations described with reference to FIG. 1D.

FIGS. 4A-4C schematically illustrate example normalized signal intensities that may be obtained using the raw signal intensities described with reference to FIGS. 2A-2C or 3A-3C.

FIG. 5 schematically illustrates base calls under different scenarios.

FIG. 6 illustrates an example of operations in a method for characterizing analytes in a sample using normalized signals.

FIGS. 7A-7B illustrate example raw signal intensities that were obtained using compositions and operations described with reference to FIGS. 1C and 1D.

FIGS. 8A-8B respectively illustrate example mean coefficients of variation (CV) based on raw and normalized signal intensities obtained using an Illumina iSeq instrument in a manner such as described with reference to FIGS. 1C-1D and 7A-7B.

FIGS. 9A-9B respectively illustrate example mean CV based on raw and normalized signal intensities obtained using an Illumina iSeq instrument in a manner such as described with reference to FIGS. 1C-1D, 7A-7B, and 8A-8B.

FIG. 10 illustrates example mean coefficients of variation based on raw and normalized signal intensities obtained using an Illumina iSeq instrument in a manner such as described with reference to FIGS. 1C-1E, 7A-7B, 8A-8B, and 9A-9B.

### DETAILED DESCRIPTION

The present application relates to characterizing analytes in a sample using normalized signals.

For example, bead-based genomics assays may suffer from the problem of signal variability that may arise from a combination of various different effects. For example, it may be desired to detect analytes, such as single nucleotide polymorphism (SNPs) or methylated bases, within a particular sample. It may be desired to use the signal intensity to detect the presence of analyte, e.g., by using the signal intensity to make a base call. Additionally, it may be desired to use the signal intensity to determine the amount of analyte within the sample, e.g., by comparing intensities for different analytes to one another. However, the raw signal intensities may vary for reasons that are not directly related to the presence or amount of a particular analyte, and such reasons thus may make it difficult to characterize that analyte, e.g., to detect the presence of the analyte or the amount of the analyte. For example, the position of a particular bead within the well of a flowcell may increase or decrease the signal. Additionally, or alternatively, a surface characteristic of the well may increase or decrease the signal. Additionally, or alternatively, a loading condition of the bead within the well may increase or decrease the signal. Additionally, or alternatively, a capture efficiency of the bead within the well may increase or decrease the signal. Additionally, or alternatively, the size of the bead may increase or decrease the signal. Additionally, or alternatively, a number of polynucleotides that are attached to the bead (e.g., to which a target polynucleotide may hybridize) may increase or decrease the signal. The resulting raw intensity differences caused by these or other factors may obscure the desired measurements, such as intensity variations that arise from analytes being present in certain amounts in a sample, such as target polynucleotides having different sequences than one another (e.g., different SNPs, or differently methylated bases at a particular location) being present in certain amounts in a sample.

As described in greater detail herein, the present subject matter solves such problems by using normalized signals. More specifically, the raw signal intensity from a first labeled nucleotide, which is directly or indirectly coupled to a substrate, is normalized using the raw signal intensity from a second labeled nucleotide, which is directly or indirectly or indirectly coupled to the same substrate before or after the first labeled nucleotide. For example, the second labeled nucleotide may be added to the same polynucleotide as the first labeled nucleotide is added, or may be added to a different polynucleotide than the first labeled nucleotide. It will be appreciated that which of the labeled nucleotides is considered to be “first” and which is considered “second” is arbitrary. It may be expected that the raw signal intensity from the first and second labeled nucleotides are likely to vary similarly as one another as a function of the position of the substrate, e.g., of the particular bead within the well of a flowcell. Additionally, or alternatively, it may be expected that the raw signal intensity from the first and second labeled nucleotides are likely to vary similarly as one another as a function of a surface characteristic of the substrate, e.g., of a well within which the particular bead is located. Additionally, or alternatively, it may be expected that the raw signal intensity from the first and second labeled nucleotides are likely to vary similarly as one another as a function of the loading condition of the substrate, e.g., of the bead within the well. Additionally, or alternatively, it may be expected that the raw signal intensity from the first and second labeled nucleotides are likely to vary similarly as one another as a function of the capture efficiency of the substrate, e.g., of the bead within the well. Additionally, or alternatively, it may be expected that the raw signal intensity from the first and second labeled nucleotides are likely to vary similarly as one another as a function of the size of the substrate, e.g., of the bead. Additionally, or alternatively, it may be expected that the raw signal intensity from the first and second labeled nucleotides are likely to vary similarly as one another as a function of the number of polynucleotides that are attached to the substrate, e.g., to the bead. Accordingly, normalizing the raw signal intensity from the first labeled nucleotide using the raw signal intensity from the second labeled nucleotide may adjust the signal intensity from the first labeled nucleotide in such a way that reduces or eliminates signal contributions from any factors (such as, but not limited to, those described herein) that approximately equally affect the raw signal intensities from both labeled nucleotides. As such, the normalized signal from the first labeled nucleotide may be expected to more accurately correlate to the presence and/or amount of an analyte.

Some terms used herein will be briefly explained. Then, some example compositions and example methods for characterizing polynucleotides in a sample using normalized signals will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially”, “approximately”, and “about” used throughout this Specification are used to describe and account for small fluctuations, such as due to variations in processing. For example, they can refer to less than or equal to ±5%, such as less than or equal to ±2%, such as less than or equal to #1%, such as less than or equal to ±0.5%, such as less than or equal to ±0.2%, such as less than or equal to ±0.1%, such as less than or equal to ±0.05%.

As used herein, “analyte” is intended to mean a chemical element that is desired to be detected. An analyte may be referred to as a “target.” Analytes may include nucleotide analytes and non-nucleotide analytes. Nucleotide analytes may include one or more nucleotides. Non-nucleotide analytes may include chemical entities that are not nucleotides. An example nucleotide analyte is a DNA analyte, which includes a deoxyribonucleotide or modified deoxyribonucleotide. DNA analytes may include any DNA sequence or feature that may be of interest for detection, such as single nucleotide polymorphisms or DNA methylation. Another example nucleotide analyte is an RNA analyte, which includes a ribonucleotide or modified ribonucleotide. RNA analytes may include any RNA sequence or feature that may be of interest for detection, such as the presence or amount of mRNA or of cDNA. An example non-nucleotide analyte is a protein analyte. A protein includes a sequence of polypeptides that are folded into a structure. Another example non-nucleotide analyte is a metabolite analyte. A metabolite analyte is a chemical element that is formed or used during metabolism. Additional example analytes include, but are not limited to, carbohydrates, fatty acids, sugars (such as glucose), amino acids, nucleosides, neurotransmitters, phospholipids, and heavy metals. In the present disclosure, analytes may be detected in the context of any suitable application(s), such as analyzing a disease state, analyzing metabolic health, analyzing a microbiome, analyzing drug interaction, analyzing drug response, analyzing toxicity, or analyzing infectious disease. Illustratively, metabolites can include chemical elements that are upregulated or downregulated in response to disease. Nonlimiting examples of analytes include kinases, serine hydrolases, metalloproteases, disease-specific biomarkers such as antigens for specific diseases, and glucose.

As used herein, elements being “different” is intended to mean that one of the elements has at least one variation relative to the other element that renders the elements distinguishable one another. For example, nucleotide analytes that are different than one another may have nucleotide sequences that vary relative to another by at least one nucleotide. As another example, proteins that are different than one another may have peptide sequences that vary relative to one another by at least one peptide. As another example, metabolites may vary relative to one another by at least one chemical group. As provided herein, different analytes can be distinguished from one another using the present systems and methods. For example, nucleotide analytes varying by at least one nucleotide relative to one another can be detected and distinguished from one another. As another example, proteins having peptide sequences varying by at least one peptide relative to one another can be detected and distinguished from one another. As another example, metabolites varying by at least one chemical group relative to one another can be detected and distinguished from one another.

As used herein, the term “nucleotide” is intended to mean a molecule that includes a sugar and at least one phosphate group, and optionally also includes a nucleobase. A nucleotide that lacks a nucleobase can be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide can be a single stranded sequence of nucleotides, such as RNA or single stranded DNA, a double stranded sequence of nucleotides, such as double stranded DNA or double stranded RNA, or can include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides can include non-naturally occurring DNA, such as enantiomeric DNA. The precise sequence of nucleotides in a polynucleotide can be known or unknown. The following are example examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, “polynucleotide” and “nucleic acid” may be used interchangeably, and can refer to a polymeric form of nucleotides of any length, such as either ribonucleotides or deoxyribonucleotides. Thus, this term includes single-, double-, or multi-stranded DNA or RNA. The term polynucleotide also refers to both double and single-stranded molecules. Examples of polynucleotides include a gene or gene fragment, genomic DNA, genomic DNA fragment, exon, intron, messenger RNA (mRNA), transfer RNA, ribosomal RNA, non-coding RNA (ncRNA) such as PIWI-interacting RNA (piRNA), small interfering RNA (siRNA), and long non-coding RNA (lncRNA), small hairpin (shRNA), small nuclear RNA (snRNA), micro RNA (miRNA), small nucleolar RNA (snoRNA) and viral RNA, ribozyme, cDNA, recombinant polynucleotide, branched polynucleotide, plasmid, vector, isolated DNA of any sequence, isolated RNA of any sequence, nucleic acid probe, primer or amplified copy of any of the foregoing. A polynucleotide can include modified nucleotides, such as methylated nucleotides and nucleotide analogs including nucleotides with non-natural bases, nucleotides with modified natural bases such as aza- or deaza-purines. In some examples, a polynucleotide can be composed of a specific sequence of four nucleotide bases: adenine (A); cytosine (C); guanine (G); and thymine (T). Uracil (U) can also be present, for example, as a natural replacement for thymine when the polynucleotide is RNA. Uracil can also be used in DNA. Thus, the term ‘sequence’ refers to the alphabetical representation of a polynucleotide or any nucleic acid molecule, including natural and non-natural bases.

As used herein, “target nucleic acid,” “target polynucleotide,” or grammatical equivalent thereof can refer to nucleic acid molecules or sequences that it is desired to identify, sequence, analyze and/or further manipulate. In some examples, a target nucleic acid can include a single nucleotide polymorphism (SNP) to be identified. In some examples, a SNP can be identified by hybridizing a first polynucleotide to a second polynucleotide including the target nucleic acid, and extending the first polynucleotide using the sequence of the second polynucleotide. Target nucleic acids and target polynucleotide are nonlimiting examples of analytes.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

By “capture” it is meant to become coupled to an analyte that is in solution. The element that performs the capturing may also be in solution, or may be coupled to a substrate.

As used herein, “hybridize” is intended to mean noncovalently attaching a first polynucleotide to a second polynucleotide along the lengths of those polynucleotides via specific hydrogen bonding pairing of nucleotide bases. The strength of the attachment between the first and second polynucleotides increases with the length and complementarity between the sequences of monomer units within those polymers. For example, the strength of the attachment between a first polynucleotide and a second polynucleotide increases with the complementarity between the sequences of nucleotides within those polynucleotides, and with the length of that complementarity. By “temporarily hybridized” it is meant that polymer sequences are hybridized to each other at a first time, and dehybridized from one another at a second time.

For example, as used herein, “hybridization”, “hybridizing” or grammatical equivalent thereof, can refer to a reaction in which one or more polynucleotides react to form a complex that is formed at least in part via hydrogen bonding between the bases of the nucleotide residues. The hydrogen bonding can occur by Watson-Crick base pairing, Hoogstein binding, or in any other sequence-specific manner. The complex can have two strands forming a duplex structure, three or more strands forming a multi-stranded complex, a single self-hybridizing strand, or any combination of thereof. The strands can also be cross-linked or otherwise joined by forces in addition to hydrogen bonding.

As used herein, a “polymerase” is intended to mean an enzyme having an active site that assembles polynucleotides by polymerizing nucleotides into polynucleotides. A polymerase can bind a primed single stranded polynucleotide template, and can sequentially add nucleotides to the growing primer to form a polynucleotide having a sequence that is complementary to that of the template.

As used herein, the term “primer” is defined as a polynucleotide having a single strand with a free 3′ OH group. A primer can also have a modification at the 5′ terminus to allow a coupling reaction or to couple the primer to another moiety. The primer length can be any number of bases long and can include a variety of non-natural nucleotides. A primer can be blocked at the 3′ end to inhibit polymerization until the block is removed.

As used herein, “extending”, “extension” or any grammatical equivalents thereof can refer to the addition of nucleotides (such as dNTPs) to a primer, polynucleotide or other nucleic acid molecule using an extension enzyme such as a polymerase, or ligase.

As used herein, the term “label” is intended to mean a structure that is coupled to an element and based upon which the presence of an element can be detected. A label may include a fluorophore, or may include a moiety to which a fluorophore may be coupled directly or indirectly. As such, a “labeled nucleotide” refers to a nucleotide that is coupled to a label.

As used herein, the term “fluorophore” is intended to mean a molecule that emits light at a first wavelength responsive to excitation with light at a second wavelength that is different from the first wavelength. The light emitted by a fluorophore may be referred to as “fluorescence” and may be detected by suitable optical circuitry.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell, or a bead located in a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA). Beads may be located in a flow cell.

As used herein, “surface” can refer to a part of a substrate or support structure that is accessible to contact with reagents, substrates (such as beads), or analytes. The surface can be substantially flat or planar. Alternatively, the surface can be rounded or contoured. Example contours that can be included on a surface are wells, depressions, pillars, ridges, channels or the like. Example materials that can be used as a substrate or support structure include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon; carbon-fibre; metal; inorganic glass; optical fibre bundle, or a variety of other polymers. A single material or mixture of several different materials can form a surface useful in certain examples. In some examples, a surface comprises wells. In some examples, a support structure can include one or more layers. Example support structures can include a chip, a film, a multi-well plate, and a flow-cell.

As used herein, “bead” can refer to a small body made of a solid material. The material of the bead may be rigid or semi-rigid. The body can have a shape characterized, for example, as a sphere, oval, microsphere, or other recognized particle shape whether having regular or irregular dimensions. In some examples, a bead or a plurality of beads can comprise a surface. Example materials that are useful for beads include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon; carbon-fiber; metal; inorganic glass; or a variety of other polymers. Example beads include controlled pore glass beads, paramagnetic beads, thoria sol, Sepharose beads, nanocrystals and others known in the art. Beads can be made of biological or non-biological materials. Magnetic beads are particularly useful due to the ease of manipulation of magnetic beads using magnets at various processes of the methods described herein. Beads used in certain examples can have a diameter, width or length from about 5.0 nm to about 100 μm, e.g., from about 10 nm to about 100 μm, e.g., from about 50 nm to about 50 μm, e.g., from about 100 nm to about 500 nm. In some examples, beads used in certain examples can have a diameter, width or length less than about 100 μm, 50 μm, 10 μm, 5 μm, 1 μm, 0.5 μm, 100 nm, 50 nm, 10 nm, 5 nm, 1 nm, 0.5 nm, 100 μm, or any diameter, width or length within a range of any two of the foregoing diameters, widths or lengths. Bead size can be selected to have reduced size, and hence get more features per unit area, whilst maintaining sufficient signal (template copies per feature) in order to analyze the features.

In some examples, polynucleotides may be coupled to beads. In some examples, the beads can be distributed into wells on the surface of a substrate, such as a flow cell. Example bead arrays that can be used in certain examples include randomly ordered BEADARRAY technology (Illumina Inc., San Diego CA). Such bead arrays are disclosed in Michael et al., Anal Chem 70, 1242-8 (1998); Walt, Science 287, 451-2 (2000); Fan et al., Cold Spring Harb Symp Quant Biol 68:69-78 (2003); Gunderson et al., Nat Genet 37:549-54 (2005); Bibikova et al. Am J Pathol 165:1799-807 (2004); Fan et al., Genome Res 14:878-85 (2004); Kuhn et al., Genome Res 14:2347-56 (2004); Yeakley et al., Nat Biotechnol 20:353-8 (2002); and Bibikova et al., Genome Res 16:383-93 (2006), each of which is incorporated by reference in its entirety.

As used herein, a “polymer” refers to a molecule including a chain of many subunits that are coupled to one another and that may be referred to as monomers. The subunits may repeat, or may differ from one another. Polymers can be biological or synthetic polymers. Example biological polymers that suitably can be included within a bridge or a label include polynucleotides, polypeptides, polysaccharides, polynucleotide analogs, and polypeptide analogs. Example polynucleotides and polynucleotide analogs suitable for use in a bridge or a label include DNA, enantiomeric DNA, RNA, PNA (peptide-nucleic acid), morpholinos, and LNA (locked nucleic acid). Polymers may include spacer phosphoramidites, which may be coupled to polynucleotides but which lack nucleobases, such as commercially available from Glen Research (Sterling, VA). Example synthetic polypeptides can include charged or neutral amino acids as well as hydrophilic and hydrophobic residues. Example synthetic polymers that suitably can be included within a bridge or label include PEG (polyethylene glycol), PPG (polypropylene glycol), PVA (polyvinyl alcohol), PE (polyethylene), LDPE (low density polyethylene), HDPE (high density polyethylene), polypropylene, PVC (polyvinyl chloride), PS (polystyrene), NYLON (aliphatic polyamides), TEFLON® (tetrafluoroethylene), thermoplastic polyurethanes, polyaldehydes, polyolefins, poly(ethylene oxides), poly(@)-alkenoic acid esters), poly(alkyl methacrylates), and other polymeric chemical and biological linkers such as described in Hermanson, Bioconjugate Techniques, third edition, Academic Press, London (2013). Synthetic polymers may be conductive, semiconductive, or insulating.

As used herein, to “detect” a signal is intended to meant to generate an electrical signal based on a label, and to determine, using the electrical signal, that the label was present. For example, as used herein, to “detect” fluorescence is intended to mean to receive light from a fluorophore, to generate an electrical signal based on the received light, and to determine, using the electrical signal, that light was received from the fluorophore. Fluorescence may be detected using any suitable optical detection circuitry, which may include an optical detector to generate an electrical signal based on the light received from the fluorophore, and electronic circuitry to determine, using the electrical signal, that light was received from the fluorophore. As one example, the optical detector may include an active-pixel sensor (APS) including an array of amplified photodetectors configured to generate an electrical signal based on light received by the photodetectors. APSs may be based on complementary metal oxide semiconductor (CMOS) technology known in the art. CMOS-based detectors may include field effect transistors (FETs), e.g., metal oxide semiconductor field effect transistors (MOSFETs). In particular examples, a CMOS imager having a single-photon avalanche diode (CMOS-SPAD) may be used, for example, to perform fluorescence lifetime imaging (FLIM). In other examples, the optical detector may include a photodiode, such as an avalanche photodiode, charge-coupled device (CCD), cryogenic photon detector, reverse-biased light emitting diode (LED), photoresistor, phototransistor, photovoltaic cell, photomultiplier tube (PMT), quantum dot photoconductor or photodiode, or the like. The optical detection circuitry further may include any suitable combination of hardware and software in operable communication with the optical detector so as to receive the electrical signal therefrom, and configured to detect the fluorescence based on such signal, e.g., based on the optical detector detecting light from the fluorophore. For example, the electronic circuitry may include a memory and a processor coupled to the memory. The memory may store instructions for causing the processor to receive the signal from the optical detector and to detect the fluorophore using such signal. For example, the instructions can cause the processor to determine, using the signal from the optical detector, that fluorescence is emitted within the field of view of the optical detector and to determine, using such determination, that a fluorophore is present. The instructions also may cause the processor to correlate the presence of the fluorophore to the presence of an analyte in the sample being characterized.

To “measure” a signal is intended to mean to determine a relative or absolute intensity of a detected signal. For example, labels coupled to different substrates, or to the same substrate, may generate the same or different intensities of signal as one another. A “raw” signal intensity refers to the absolute intensity of the signal that is detected prior to adjustment (e.g., normalization) of that signal using a calculation that is particular to that signal. Accordingly, to “measure” fluorescence is intended to mean to determine a relative or absolute intensity of the fluorescence that is detected. For example, fluorophores coupled to different substrates, or to the same substrate, may emit the same or different intensities of fluorescence as one another. A “raw” fluorescence intensity refers to the absolute intensity of the fluorescence that is detected prior to adjustment (e.g., normalization) of that signal using a calculation that is particular to that signal. A signal still may be considered “raw” even if processed using signal conditioning circuitry, such as amplifiers, filters, analog-to-digital conversion, or the like, because all signals may be similarly processed.

“Normalizing” a signal intensity is intended to refer to adjusting a signal intensity using a different signal intensity. For example, a signal intensity from a first labeled nucleotide may be normalized using a signal intensity from a second labeled nucleotide. Illustratively, the signal intensity from the first labeled nucleotide may be divided by the signal intensity from a second labeled nucleotide. It will be appreciated that the signal intensity which is adjusted may be, but need not necessarily, be a raw signal intensity.

To “characterize” an analyte is intended to refer to detecting the identity of the analyte, to determine the amount of the analyte, or to detect both the identity and determine the amount of the analyte.

### Characterizing Analytes in a Sample Using Normalized Signals

The present application relates to characterizing analytes in a sample using normalized signals.

For example, signal precision is useful in bead-based genomics assays for applications such as genotyping, non-invasive prenatal testing (NIPT), DNA methylation, or assays such as described in PCT Publication No. WO2021/074087, the entire contents of which are incorporated by reference herein. These applications may be intended to detect relatively small variations in signal intensities from a given sample, or from different samples. For example, NIPT may be intended to detect variations in signals from a parental DNA library that is mixed with a relatively small amount of fetus DNA. Similarly, DNA methylation detection may be intended to detect variations in signals from methylated, or non-methylated, SNPs in a partially methylated DNA sample. For genotyping, it may be intended to detect clustering of expected SNP types. Genotyping, NIPT, methylation detection, and assays such as described in PCT Publication No. WO2021/074087 may offer practical and scalable sample plexity, platform flexibility, alignment of genotyping and sequencing technologies, and relatively low cost at low sample numbers. However, as described in greater detail below elsewhere herein, signal variability within or between beads may make it difficult to detect the intended variations in signal intensities arising from the analytes themselves.

For example, beads may be coupled to specific oligonucleotides for capturing respective analytes within a sample, such as target polynucleotides within a target library. Single base extension (SBE) then may be used in which a labeled nucleotide is used to extend the oligonucleotide, and then is detected. It may be intended to identify the labeled nucleotide that became coupled to a bead, and to detect the identity of the analyte based on the identified labeled nucleotide. In some circumstances it also may be intended to determine the amount of labeled nucleotide that became coupled to the bead, and to determine the amount of the analyte within the sample based on the determined amount of labeled nucleotide. However, the protocols used to hybridize the analytes (e.g., target polynucleotides) to the beads' respective oligonucleotides, perform SBE, and/or perform other processes may lead to variability in the intensity of signals from beads, and thus may detrimentally affect the analyte's characterization. Additionally, or alternatively, signal variability may arise from non-uniform bead capture on a surface at which bead orientation affects the signal, such as a CMOS sensor on which wells are disposed. Such non-uniform bead capture may occur, for example, depending on the efficiency of the bead loading procedure and/or any size differences among the beads and/or the wells.

Methods are provided herein for reducing variability in signal intensity for extension-based assays by normalizing the signal using one or more additional cycles of labeled nucleotide addition for the same substrate. In some examples, the labeled nucleotide used for the normalization may be added to the same oligonucleotide as the labeled nucleotide used for characterizing the analyte, while in other examples the labeled nucleotide used for the normalization may be added to a different oligonucleotide that is coupled to the same substrate. The additional cycle(s) of labeled nucleotide addition may also or additionally serve as an error correction mechanism in case the additional cycle(s) yield unexpected base calls. For example, if it is known that the locus after a SNP should be a particular base but a different base is detected, then the signal from that SNP may be discarded because a non-target polynucleotide may have been captured. While certain examples herein may relate to normalizing fluorescent signals obtained using genotyping assays performed using beads on a sequencer, it should be appreciated that the present subject matter may be used to normalize any type of signals obtained using any suitable type of assay.

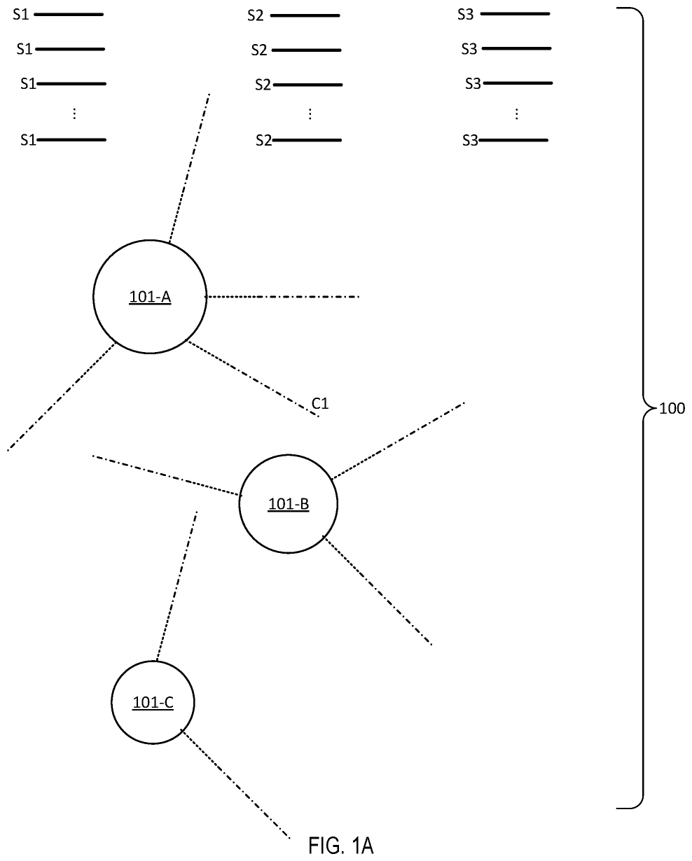

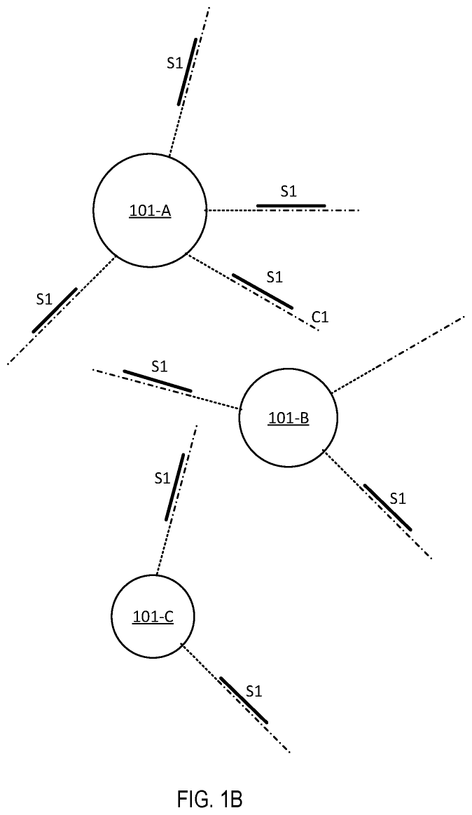

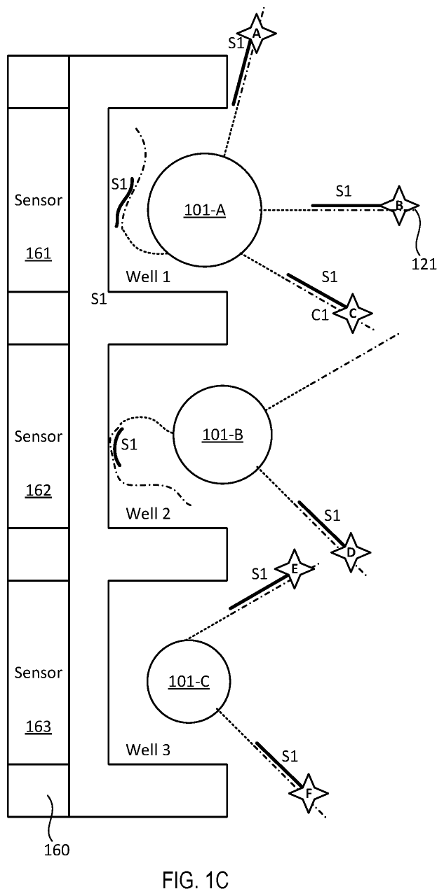

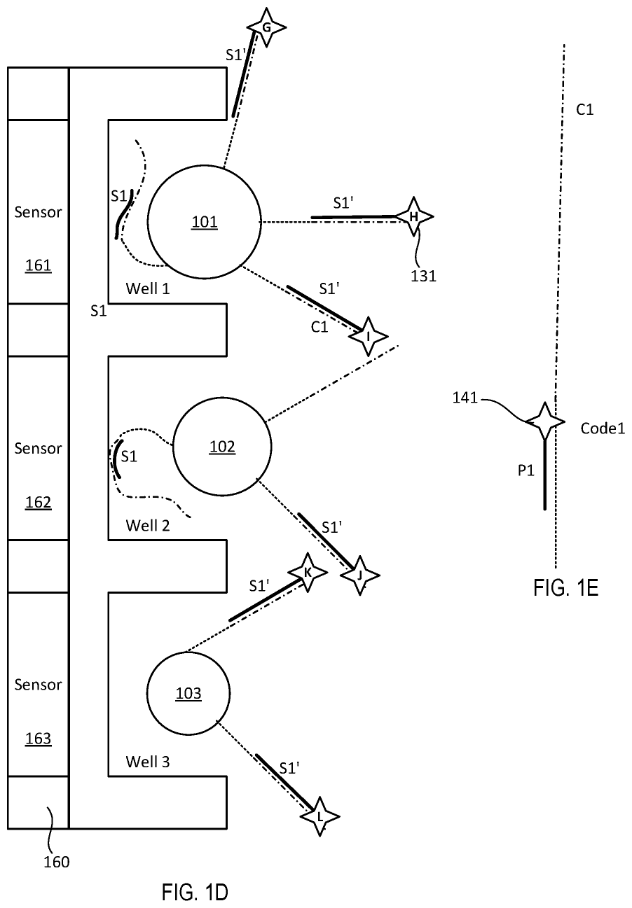

FIGS. 1A-1E schematically illustrate example compositions and operations used to characterize analytes in a sample using normalized signals. Referring first to FIG. 1A, composition 100 may include a fluid (not specifically illustrated) in which a sample including one or more analytes, e.g., a plurality of target polynucleotides S1, S2, S3, is contacted with a plurality of beads 101-A, 101-B, 101-C. It may be desired to determine the amount within the sample of at least one of the analytes. For example, the sample may include a plurality of each of target polynucleotides S1, S2, S3, and it may be desired to determine the absolute amount of at least one of the target polynucleotides, or the relative amount of two or more of the target polynucleotides as compared to one another, within the sample. In this simplified example, target polynucleotides S1, S2, and S3 are present in the sample for illustrative purposes. Different beads may be provided that respectively are specific to an analyte that may be present in the sample. In this simplified example, each of beads 101-A, 101-B, and 101-C includes a plurality of capture oligonucleotides C1 that are substantially complementary to target polynucleotide S1, and thus specific to that target polynucleotide (analyte). Other beads (not specifically illustrated) may include respective pluralities of other capture oligonucleotides that are substantially complementary to other target polynucleotides, e.g., target polynucleotides S2 and S3. Because of manufacturing variability, beads 101-A, 101-B, 101-C may be different sizes than one another, and/or may include different numbers of oligonucleotides than one another. In this simplified example, bead 101-A is the largest bead and includes four oligonucleotides C1, bead 101-B is smaller and includes three oligonucleotides C1, and bead 101-A is still smaller and includes two oligonucleotides C1, for illustrative purposes. It will be appreciated that each bead may include tens, hundreds, thousands, or more of oligonucleotides C1, that any suitable number of beads with such oligonucleotides may be provided, and that different beads may include other oligonucleotides that are substantially complementary to other, different target polynucleotides. Further details regarding oligonucleotides C1 are provided below with reference to FIG. 1E. In a manner such as illustrated in FIG. 1B, polynucleotides S1 respectively may hybridize to oligonucleotides C1 respectively coupled to beads 101-A, 101-B, and 101-C. It is desirable that the amount of each polynucleotide (or other analyte) that becomes coupled to the respective bead is proportional to the amount of that polynucleotide in the sample, so as to facilitate characterization of that polynucleotide (e.g., absolute quantitation of that polynucleotide, or quantitation of that polynucleotide relative to one or more other polynucleotides within the same sample). In this simplified example, if there were no variability between beads, then substantially equal amounts of target polynucleotide S1 would become hybridized to equal numbers of the respective oligonucleotides which would coupled in equal numbers to each of those beads. However, because beads 101-A, 101-B, 101-C are different sizes and include different numbers of oligonucleotide C1, the numbers of polynucleotides S1 that respectively become hybridized to those beads may not be proportional to the amount of that polynucleotide in the sample. Furthermore, hybridization kinetics and hairpin formation also or alternatively may cause the numbers of polynucleotides that hybridize to respective beads to vary from the amount of the polynucleotides in the sample. In the simplified example shown in FIG. 1B, while all four oligonucleotides on bead 101-A may capture corresponding polynucleotides S1, due to hybridization kinetics or hairpin formation only a subset of the already different number of oligonucleotides on bead 101-B may capture corresponding polynucleotides S1, and a reduced number of oligonucleotides on bead 101-C are even available to capture polynucleotides S1. Accordingly, not all of polynucleotides S1 may be captured from solution, and different numbers of polynucleotides S1 may be captured by different beads.

The beads may be captured on a substrate surface, e.g., may become coupled to a particular region of the substrate surface at which the beads subsequently are imaged to obtain a fluorescence intensity therefrom. In some examples, such as illustrated in FIG. 1C, beads 101-A, 101-B, 101-C may be captured within respective wells 1, 2, and 3. For example, beads 101-A, 101-B, 101-C each may include oligonucleotides (not specifically illustrated) that hybridize to other oligonucleotides (not specifically illustrated) within the respective wells. In other examples, beads 101-A, 101-B, 101-C may be electrostatically attracted to respective wells. In still other examples, beads 101-A, 101-B, 101-C may be paramagnetic, and may be magnetically attracted to respective wells. However, it will be appreciated that beads 101-A, 101-B, 101-C suitably may be captured on any suitable substrate surface, such as a shaped or substantially planar surface that is located within a flowcell. Additionally, or alternatively, beads 101-A, 101-B, 101-C may be contacted with a substrate (e.g., disposed within respective wells) before being contacted with a fluid that includes analytes, e.g., target polynucleotides S1, S2, S3. If wells are used, wells 1, 2, and 3 optionally may include sensors 161, 162, 163 from capturing the fluorescence intensity from respective beads 101-A, 101-B, and 101-C. However, other suitable detection circuitry may be used to capture such fluorescence intensities, such as an optical sensor configured to collect light from each of the beads coupled to the substrate surface.

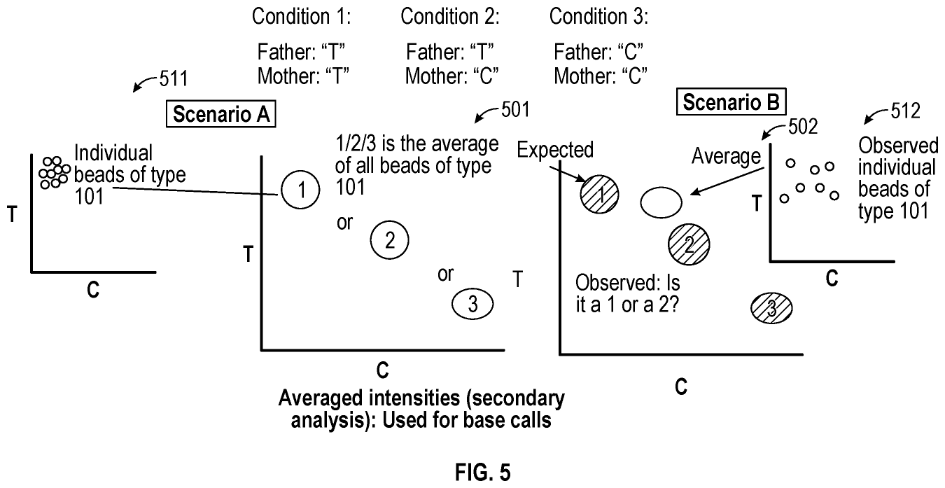

After the beads are captured at the substrate surface (e.g., within wells), in a manner such as also illustrated in FIG. 1C, labeled nucleotides 121 may be added to oligonucleotides C1 using the sequence of polynucleotides S1. It should be appreciated that the base of the particular labeled nucleotides added to respective oligonucleotides C1 will depend on the next base in the sequence of the corresponding polynucleotide S1. For example, if polynucleotides S1 are all identical to one another, then the same type of labeled polynucleotide 121 may be added to oligonucleotides C1. On the other hand, if polynucleotides S1 include different sequences than one another, e.g., include a heterozygous single nucleotide polymorphism (SNP), then different types of polynucleotides may be added to different ones of the oligonucleotides, e.g., in a manner such as described below with reference to FIG. 5. The different types of nucleotides that are added to different beads may be distinguished from one another using, for example, different types of fluorescent labels in a manner such as known in the art.

A signal intensity then may be measured from each of the beads. For example, labeled nucleotides 121 may include respective fluorophores, and suitable detection circuitry detects the intensity of fluorescence (which may be proportional to the number of fluorescent photons emitted responsive to optical excitation at a suitable wavelength) from respective ones of the beads. Such detection circuitry may, for example, include a CMOS image sensor 160 including sensors 161, 162, 163 respectively positioned to measure the intensity of fluorescence from the beads respectively within each of wells 1, 2, and 3. However, any suitable detection circuitry may be used to measure signals from the beads, such as an optical sensor that measures the intensity of fluorescence received from each of the respective beads through an optical component, such as a microscope objective. The detection circuitry may be configured to measure the intensity of fluorescence from individually labeled nucleotides, or may be configured to measure the total intensity of fluorescence from the collection of labeled nucleotides that are coupled to a given bead.

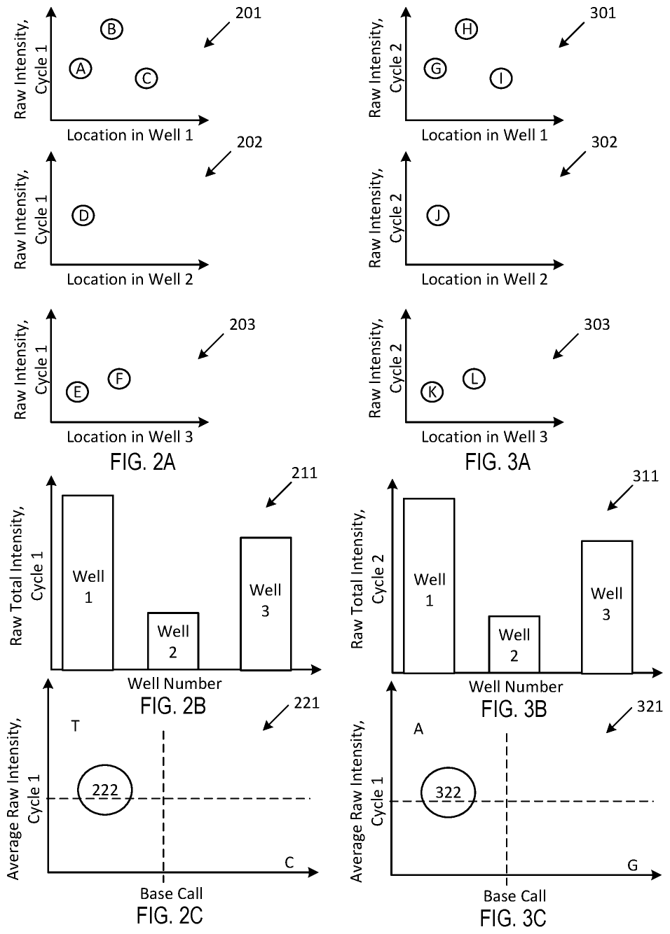

The signals from the labeled nucleotides 121 that are coupled to beads 101-A, 101-B, 101-C may have different intensities than one another, depending on a variety of factors such as described herein. For example, FIGS. 2A-2C schematically illustrate example raw signal intensities that may be obtained using the compositions and operations described with reference to FIG. 1C. Although the detection circuitry may not necessarily measure the fluorescence from individual labeled nucleotides coupled to a particular bead, a brief discussion of potential causes for differences in fluorescence from individual labeled nucleotides may be helpful to understand potential causes for differences in total fluorescence from different beads, as well as to understand the manner in which such fluorescence may be normalized. Plot 201 illustrated in FIG. 2A illustrates example raw signal intensities from each of labeled nucleotides 121 (here labeled A, B, and C, corresponding to such labels in FIG. 1C) as well as their respective locations in well 1. It may be seen that labeled nucleotides A and C have relatively low raw signal intensity, while labeled nucleotide B has somewhat higher raw signal intensity. Plot 202 illustrated in FIG. 2A illustrates example raw signal intensities from labeled nucleotide 121 (here labeled D corresponding to such label in FIG. 1C) as well its location in well 2. It may be seen that labeled nucleotide D has a relatively low raw signal intensity. Plot 203 illustrated in FIG. 2A illustrates example raw signal intensities from each of labeled nucleotides 121 (here labeled E and F corresponding to such labels in FIG. 1C) as well as their location in well 3. It may be seen that labeled nucleotide E has a relatively low raw signal intensity, while labeled nucleotide F has a somewhat higher raw signal intensity.

Plot 211 in FIG. 2B illustrates example raw total signal intensities from wells 1, 2, and 3 for this first cycle (“Cycle 1”), which in some examples may correspond to the signal actually measured by the detection circuitry, e.g., if the detection circuitry does not individually distinguish the labeled nucleotides from one another. From FIG. 2B, it may be seen that the raw total intensity from well 1 is significantly greater than the raw total intensity from well 2, and that the raw total intensity from well 3 is greater than that from well 2 and lower than that from well 1. The raw total signal intensities from each of wells 1, 2, and 3 may be suitably averaged for use in making a base call. For example, plot 221 in FIG. 2C illustrates the average 222 of the raw total signal intensities from wells 1, 2, and 3, overlaid with an example scheme for making base calls (e.g., between T and C). If an average raw total signal falls within the quadrant corresponding to one of those bases, e.g., T, then software calls nucleotide 121 as that base. As shown in FIG. 2C, because the average raw total signal intensity 222 from wells 1, 2, and 3 is relatively low, it may not necessarily fall entirely within the appropriate quadrant, thus potentially leading to difficulty or error in making the base call. In this simplified example, all of the nucleotides 121 are of the same type, similar to condition 1 described below with reference to FIG. 5.

Note that labeled nucleotides 121 may be configured to emit substantially the same amount of fluorescence as each other if within the same physical and chemical environment. However, in an actual system, such environments may differ from one another depending on the particular location of the labeled nucleotide relative to the respective bead 101-A, 101-B, or 101-C, relative to the substrate (e.g., relative to one or more features within the respective well 1, 2, or 3), and relative to one another. Additionally, detection circuitry (e.g., CMOS sensor 160) may collect different fluorescence intensities from different ones of labeled nucleotides, e.g., depending on the particular location of the labeled nucleotide relative to the respective sensor 161, 162, 163, if such sensors are used. Accordingly, whereas in a hypothetical, perfectly controlled system, labeled nucleotides 121 may generate fluorescence of equal intensities that are detected equally by the detection circuitry, in an actual system the amount of fluorescence that is emitted by, and measured from, the labeled nucleotides may vary greatly. Additionally, or alternatively, any combination of differences between the beads, interactions between capture oligonucleotides (e.g., C1) and target polynucleotides (e.g., S1), or interactions between the beads and the substrate, may cause variations in fluorescence intensity or detection. For example, different numbers of capture oligonucleotides (e.g., C1) may be coupled to respective beads 101-A, 101-B, 101-C, thus potentially resulting in different raw signal intensities from each of the beads. Or, for example, different ones of the capture oligonucleotides may capture target polynucleotides with different efficiencies, such as may arise from different hybridization kinetics, non-specific binding, hairpin formation, or the like. Or, for example, different ones of the duplexes between a capture oligonucleotide and its corresponding target polynucleotide may not necessarily be at a location at which a labeled nucleotide may be added. Or, for example, beads may be located or oriented differently relative to the detection circuitry which may cause otherwise similar beads to yield different raw signal intensities.

Any combination of any such issues, some of which are intended to be represented in FIGS. 1B and 1C (e.g., different bead sizes, different capture of target polynucleotides, different capture of beads relative to the substrate, different access of labeled nucleotides to be added to capture oligonucleotides, and the like), may make it difficult to accurately characterize the analytes (e.g., target polynucleotides S1, S2, S3) in a given sample, for example in a manner such as described with reference to Scenario B of FIG. 5. For example, it may be desired to use the signals from the labeled nucleotides 121 to identify the labeled nucleotides, e.g., to determine which base became coupled to beads 101-A, 101-B, and 101-C. As another example, it may be desired to use the signal from the labeled nucleotides 121 to determine the absolute or relative amounts of analytes (e.g., target polynucleotides) in a given sample, e.g., to determine the amount of each of the analytes that became coupled to beads 101-A, 101-B, and, as is intended to be measured using the respective signals from those beads. It may be difficult to make such characterizations using raw signal intensities such as described with reference to FIGS. 2A-2C. For example, because the signals from beads 101-A, 101-B, 101-C may vary because of reasons unrelated to the identity of the base (labeled nucleotide 121), it may be difficult to make a base call. As another example, because the signals from beads 101-A, 101-B, 101-C may vary because of reasons that are not directly related to the amount of analyte in the sample, it may be difficult to determine the relative or absolute amounts of the analyte in the sample. In comparison, in a perfectly controlled system, the raw total intensities from beads of the same type, such as illustrated in FIG. 2B, would be expected to be substantially equal to one another, reflecting equivalent capture of target polynucleotides and measurement of emission from labeled nucleotides coupled thereto.

As provided herein, signal intensities from other labeled nucleotides may be used to normalize raw signal intensities that may be obtained in a manner such as described with reference to FIGS. 2A-2C, thus leading to significant improvements in the accuracy of base calling and/or of determining the amount of analytes in a sample. For example, as illustrated in FIG. 1D, labeled nucleotides 131 may be added to further extend target polynucleotides (labeled S1′ to represent that they have already been extended using labeled nucleotides 121) using the sequence of capture oligonucleotides C1. In some examples, labeled nucleotides 121 are blocked when added to polynucleotides S1, and are deblocked before adding labeled nucleotides 131. Labeled nucleotides 131 may be added to respective positions adjacent to where labeled nucleotides 121 were added. Alternatively, one or more additional nucleotides may be added to positions between where the labeled nucleotides 121 and the labeled nucleotides 131 respectively are added.

A signal intensity may be measured from each of the labeled nucleotides 131 in a manner similar to that described for labeled nucleotides 121. Labeled nucleotides 131 may, in some examples, include fluorophores. The fluorophores may fluoresce at substantially the same wavelength as one another and/or as any of the fluorophores of labeled nucleotides 121, or at different wavelengths than one another and/or than any of the fluorophores of labeled nucleotides 121.

In a manner similar to that described with reference to FIGS. 2A-2C, the signals from the labeled nucleotides 131, and more generally from beads 101-A, 101-B, 101-C, may have different intensities than one another, depending on a variety of factors such as described elsewhere herein. For example, FIGS. 3A-3C schematically illustrate example raw signal intensities that may be obtained using the compositions and operations described with reference to FIG. 1D. Plot 301 illustrated in FIG. 3A illustrates example raw signal intensities from each of labeled nucleotides 131 (here labeled G, H, and I, corresponding to such labels in FIG. 1D) as well as their respective locations in well 1. It may be seen that labeled nucleotides G and I have relatively low raw signal intensity, while labeled nucleotide H has somewhat higher raw signal intensity. Plot 302 illustrated in FIG. 3A illustrates example raw signal intensities from labeled nucleotide 131 (here labeled J corresponding to such label in FIG. 1D) as well its location in well 3. It may be seen that labeled nucleotide J has a relatively low raw signal intensity. Plot 303 illustrated in FIG. 3A illustrates example raw signal intensities from each of labeled nucleotides 131 (here labeled K and L corresponding to such labels in FIG. 1D) as well as their location in well 3. It may be seen that labeled nucleotide K has a relatively low raw signal intensity, while labeled nucleotide L has a somewhat higher raw signal intensity.

Plot 311 in FIG. 3B illustrates example raw total signal intensities from wells 1, 2, and 3 for this second cycle (“Cycle 2”), which in some examples may correspond to the signal actually measured by the detection circuitry, e.g., if the detection circuitry does not individually distinguish the labeled nucleotides from one another. From FIG. 3B, it may be seen that the raw total intensity from well 1 is significantly greater than the raw total intensity from well 2, and that the raw total intensity from well 3 is greater than that from well 2 and lower than that from well 1. The raw total signals from each of wells 1, 2, and 3 may be suitably averaged for use in making a base call. For example, plot 321 in FIG. 3C illustrates the average of the raw total signal intensities 322 from wells 1, 2, and 3, overlaid with an example scheme for making base calls (e.g., between A and G). If an average raw total signal falls within the quadrant corresponding to one of those bases, e.g., A, then software calls nucleotide 131 as that base. As shown in FIG. 3C, because the average raw total signal intensity 322 from wells 1, 2, and 3 is relatively low, it may not necessarily fall entirely within the appropriate quadrant, thus potentially leading to difficulty or error in making the base call in a manner such as described with reference to Scenario B of FIG. 5.

Indeed, it also may be seen that the raw signal intensities for labeled nucleotides G, H, and I are similar to those for labeled nucleotides A, B, and C, for example because they are similarly located relative to bead 101-A, well 1, and CMOS sensor 161 as are labeled nucleotides A, B, and C, respectively. It also may be seen that the raw signal intensities for labeled nucleotide J is similar to that for labeled nucleotide D, for example because it is similarly located relative to bead 101-B, well 2, and CMOS sensor 162 as is labeled nucleotide D. It also may be seen that the raw signal intensities for labeled nucleotides K and L are similar to those for labeled nucleotides E and F, for example because they are similarly located relative to bead 101-C, well 3, and CMOS sensor 163 as are labeled nucleotides E and F, respectively. As such, the example raw signal intensities from wells 1, 2, and 3 for this second cycle (“Cycle 2”), such as illustrated in FIG. 3B, are similar to those for the first cycle (“Cycle 1”), such as illustrated in FIG. 2B. Additionally, the average raw total intensities from those wells for this second cycle, such as illustrated in FIG. 3C, are similar to those for the first cycle, such as illustrated in FIG. 2C. Although the detection circuitry may not necessarily measure the fluorescence from individual labeled nucleotides coupled to a particular bead, it may be useful to visualize the manner in which different labeled nucleotides 121, 131 that are coupled to the same particular capture oligonucleotide C1 as one another may generate similar levels fluorescence as one another because they are in similar environments as one another, and thus may generate similar raw intensities from a particular well, as well as similar average raw total intensities from all of the wells including that type of particular capture oligonucleotide.

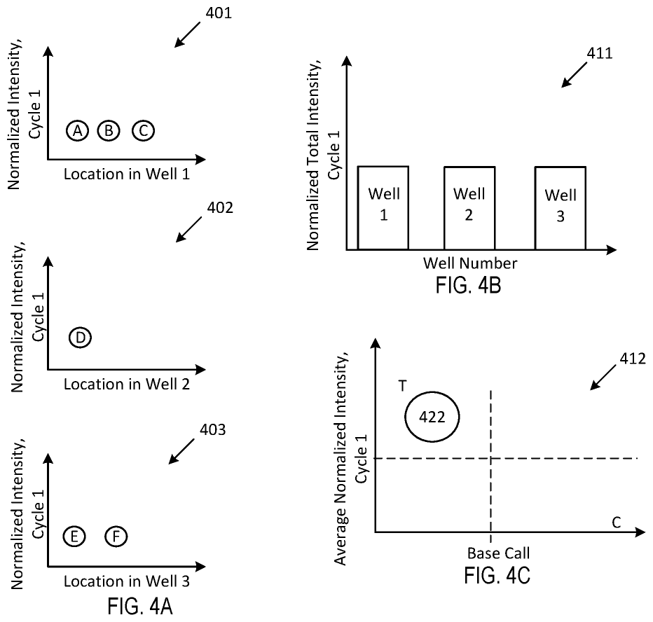

As provided herein, the raw signal intensities from labeled nucleotides in one such cycle may be used to normalize the raw signal intensities from labeled nucleotides in another such cycle, regardless of the particular temporal order in which the two cycles are performed. The normalized signal intensity may be used to characterize the analyte(s) in the sample with improved accuracy. For example, FIGS. 4A-4C schematically illustrate example normalized signal intensities that may be obtained using the raw signal intensities described with reference to FIGS. 2A2C and 3A-3C. Although the detection circuitry may not necessarily measure the intensities from individual nucleotides, the plots in FIG. 4A may provide a helpful visualization of the manner in which aggregations of such signal intensities from different labeled nucleotides, in similar environments as one another, may be mathematically related to one another in such a way as to be useful in normalizing the total intensity from an ensemble of such labeled nucleotides within a given well. More specifically, plot 401 illustrated in FIG. 4A illustrates example normalized signal intensities from each of labeled nucleotides 121 A, B, and C as well as their location in well 1. These normalized signal intensities may be generated by respectively dividing the raw signal intensity from labeled nucleotides 121 A, B, and C by the raw signal intensity from corresponding labeled nucleotides 131 G, H, and I. It may be seen that the normalized signal intensities of labeled nucleotides A, B, and C are approximately the same as one another, even though their locations within well 1 differ. Plot 402 illustrated in FIG. 4A illustrates an example normalized signal intensity from labeled nucleotide D. This normalized signal intensity may be generated by respectively dividing the raw signal intensity from labeled nucleotides 121 D by the raw signal intensity from corresponding labeled nucleotide 131 J. It may be seen that the normalized signal intensities of labeled nucleotides A, B, C, and D are approximately the same as one another, even though D is coupled to a different bead of a different size and located in a different well than A, B, and C.

Plot 403 illustrated in FIG. 4A illustrates example normalized signal intensities from each of labeled nucleotides 121 E and F. These normalized signal intensities may be generated by respectively dividing the raw signal intensity from labeled nucleotides 121 E and F by the raw signal intensity from corresponding labeled nucleotides K and L. It may be seen that the normalized signal intensities of labeled nucleotides E and F are approximately the same as one another, even though their locations within well 3 differ. It also may be seen that the normalized signal intensities of labeled nucleotides E and F are approximately the same as those of labeled nucleotides A, B, C, and D, even though they are coupled to a bead of different size and located within a different well. Accordingly, even if fluorescence is not necessarily measured from individual nucleotides, it may be understood from FIG. 4A that normalization may align the intensities from different nucleotides with one another.

In some examples, the total intensity from an ensemble of nucleotides in a first cycle may be used to normalize the total intensity from an ensemble of nucleotides in a second cycle. For example, if some beads are dim in a first cycle then they will also be dim in a second cycle (e.g., an extension cycle or a decode cycle). As such, the raw total intensity for a first cycle for each well (e.g., as illustrated in FIG. 2B) may be divided by the raw total intensity for a second cycle for each well (e.g., as illustrated in FIG. 3B) to obtain a normalized total signal intensity, such as illustrated in plot 411 in FIG. 4B, in which it may be seen that the total normalized intensity from each well 1, 2, and 3 is approximately equal. As such, in the simplified example illustrated in FIGS. 1A-1D, the normalized intensity from each of the wells may be approximately equal, even though the beads had different numbers of oligonucleotides, were of different sizes, were located differently in the respective wells, and were located differently relative to the respective CMOS sensors. Accordingly, the analytes (e.g., target polynucleotides S1, S2, and S3) may be characterized with significantly higher accuracy using the normalized signal intensities than may be achievable using the raw signal intensities. Illustratively, the nucleotides 121 may be identified (base called) with higher accuracy, e.g., resulting in a shift in the average raw intensity from the location 222 illustrated in FIG. 2C to the location 422 illustrated in plot 412 in FIG. 4C. Additionally, or alternatively, an amount of the target polynucleotides in the sample may be determined, e.g., based on the absolute value of the normalized intensity or based on a comparison of the normalized intensities to each other. Illustratively, a difference between quantities of different polynucleotides (or other analytes) may be calculated using the difference between the normalized signal intensities for bases added to the respective beads' oligonucleotides using those polynucleotides.

It will be appreciated that any suitable normalization approach may be used. In one nonlimiting example, a normalization factor may be generated by dividing the mean (average) raw intensity from all of the labeled nucleotides added to a given bead type during a second cycle (e.g., from nucleotides 131), by the raw total intensity from each of those wells during that cycle. In one nonlimiting example, the second cycle adds nucleotide G as nucleotides 131 to each of the beads. An expected value of G intensity is obtained using the mean from all beads of that intensity channel. The signal from each of the beads is normalized using the expected value of the G intensity. Illustratively, if for some reason, the G signal for a given bead is 100 as opposed to the expected (mean) of 153, a normalization factor of 1.53 is obtained. Such a factor may be used to multiply the raw total intensity from the corresponding well during a first cycle (e.g., from nucleotides 121). Accordingly, such a factor may appropriately boost the raw total intensity for each well for the first cycle, and all beads of that type similarly should have their signal from the first cycle normalized accordingly.

It further should be appreciated that the signal intensity used to perform the normalization may be from any suitable labeled nucleotides, and that such labeled nucleotides need not necessarily be added to the same polynucleotide as are the labeled nucleotides for which the signal is being normalized. For example, in a manner such as illustrated in FIG. 1E, capture oligonucleotides C1 may be coupled to barcode oligonucleotide Code1 (bead not expressly illustrated, but coupled directly or indirectly to Code1). Other types of capture oligonucleotides, not specifically illustrated, may be coupled to other barcode oligonucleotides. The sequences of the barcode oligonucleotides may correspond to the particular analytes (e.g., polynucleotide sequences S1, S2, or S3) that are characterized using the respective beads. In a manner such as illustrated in FIG. 1E, the target polynucleotide may be dehybridized from capture oligonucleotide C1, and then a primer PI may be hybridized to a corresponding primer region of the barcode oligonucleotide, and primer PI may be extended using a labeled nucleotide 141. Alternatively, a fluorescently labeled oligonucleotide that includes labeled nucleotide 141 may be hybridized to barcode oligonucleotide Code1. The signal from labeled nucleotide 141 may be used similarly as the signal from labeled nucleotides 131 in a manner such as described with reference to FIGS. 1D, 3A-3C, and 4A-4C. For example, because labeled nucleotide 141 is coupled to the same capture oligonucleotide as was labeled nucleotide 121, the two labeled nucleotides may be expected to have the same or similar environments and thus similar levels of fluorescence as one another. Accordingly, the signal intensity from labeled nucleotide 141, whether individually or from an aggregate or mean of such labeled nucleotides, may be used to normalize the signal from labeled nucleotide 121. In other examples (not specifically illustrated), the barcode oligonucleotide may be coupled to the bead separately from the capture oligonucleotide to which the barcode corresponds. For further details regarding the use and “decoding” of barcode oligonucleotides coupled to beads, see Gunderson et al., “Decoding randomly ordered DNA arrays,” Genome Research 14:870-877 (2004), the entire contents of which are incorporated by reference herein.

In some examples, it may be useful to select between using the signal from labeled nucleotide(s) 141 or from labeled nucleotide(s) 131 for use in normalizing the signal from labeled nucleotide(s) 121. For example, if the beads are approximately alike but target capture may be more significantly efficient in some as compared to the others (e.g., because of differences in hybridization kinetics, non-specific binding, hairpin formation, or the like), labeled nucleotides 131 may be expected to have a more similar environment to labeled nucleotides 121 than may labeled nucleotides 141. For example, differences in target capture may depend on the conditions of hybridization and the quality of samples, so different extension cycles from beads of the same type may be expected to yield similar raw signal intensities that may be used for normalization. In comparison, after dehybridizing the target polynucleotide S1, the hybridization of decode primers or oligonucleotides to different barcode oligonucleotides may be expected to have relatively similar hybridization kinetics and concentrations as one another that are not as closely related to the issues that caused differences in fluorescence between labeled nucleotides 121. In this circumstance, the signal from labeled nucleotides 131, added in an extension cycle, may be suitable to use for normalizing the signal from labeled nucleotides 121.

In another example, if the beads are not alike or are otherwise imperfectly prepared, e.g., some beads have more capture oligonucleotides (and corresponding barcode oligonucleotides) than other beads, e.g., in a manner such as described with reference to FIGS. 1A-1D, variation between beads may affect the addition of labeled nucleotides 121, labeled nucleotides 131, and labeled nucleotides 141 similarly as one another. Accordingly, the signal from labeled nucleotides 131, or the signal from labeled nucleotides 141, or both the signal from labeled nucleotides 131 and the signal from labeled nucleotides 141, may be suitable to use for normalizing the signal from labeled nucleotides 121.

In yet another example, if the beads are approximately alike but are located differently relative to the detection circuitry and/or features of the well, variation between beads may affect the addition of labeled nucleotides 121, labeled nucleotides 131, and labeled nucleotides 141 similarly as one another. Accordingly, the signal from labeled nucleotides 131, or the signal from labeled nucleotides 141, or both the signal from labeled nucleotides 131 and the signal from labeled nucleotides 141, may be suitable to use for normalizing the signal from labeled nucleotides 121.

It will be appreciated that a combination of these or other such issues may be present in an actual system. Accordingly, it may be useful to select between using the signal from labeled nucleotides 131, or the signal from labeled nucleotides 141, or both the signal from labeled nucleotides 131 and the signal from labeled nucleotides 141, for normalizing the signal from labeled nucleotides 121. For example, the detection circuitry may be suitably programmed to select one particular normalization strategy from among such options based upon whether the relative intensities within respective wells are more similar between the additions of nucleotides 121 and 131 (in which case nucleotides 131 are used for the normalization), or are more similar between the additions of nucleotides 121 and 141 (in which case nucleotides 141 are used for the normalization).

The present inventors recognized that the raw signal intensities from one such cycle may be used to normalize another such cycle, regardless of the particular temporal order in which the two cycles are performed and regardless of whether the cycles are performed using the same oligonucleotides, and that the normalized signal intensity may be used to characterize the analytes in the sample with improved accuracy. In one nonlimiting example, FIG. 5 schematically illustrates base calls under different scenarios. In this example, there are three possible conditions relating to a T/C SNP type at a given allele within target polynucleotides within a sample obtained from a fetus, e.g., using non-invasive prenatal testing (NIPT). In the homozygous condition (1), both the mother and the father have “T”. In the heterozygous condition (2), the mother has “C” and the father has “T”. In the homozygous condition (3), both the mother and father have “C.” It may be desired to determine which of these three conditions (1, 2, or 3) the fetus possesses. Scenario A in FIG. 5 corresponds to a “perfect” setting in which condition 1 (pure T), condition 2 (mixture of T and C), and condition 3 (pure C) are easy to differentiate because all beads of a given type for condition 1 will average to pure T, pure C, or pure T/C for a given sample. For example, as shown in inset 511 to plot 501, the raw intensities from each bead of the appropriate type give close signal to each other. Note that each data point shown in the inset corresponds to the raw intensity from a bead that is attached to multiple target oligonucleotides, and to which multiple labeled nucleotides have been coupled in a manner such as described with reference to FIG. 1C. Scenario B in FIG. 5 corresponds to a “real world” setting in which differences within or between beads, such as described elsewhere herein, may cause differences in the raw intensity from different beads as shown in inset 512 to plot 502. These differences may make it harder to make a base call for use in differentiating, for example, between condition 1 or condition 2. As provided herein, the present normalization operations may be used to normalize raw intensities, such as shown in inset 512, to obtain normalized intensities that are clustered in a manner such as shown in inset 511 and thus may be used to make more accurate base calls.

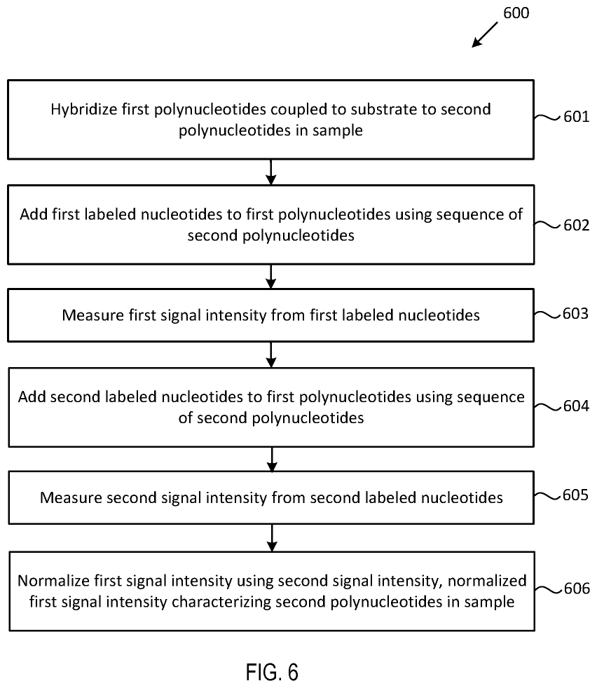

It will be appreciated that any suitable combination of operations may be used to normalize signals in a manner such as described with reference to FIGS. 1A-1E, 2A-2B, 3A-3B, 4A-4B, and 5. For example, FIG. 6 illustrates an example of operations in a method for characterizing analytes in a sample using normalized signals. Method 600 illustrated in FIG. 6 may include hybridizing first polynucleotides coupled to a first substrate to second polynucleotides in a sample (operation 601). For example, first polynucleotides C1 coupled to bead 101-A may be hybridized to second polynucleotides S1 in a sample in a manner such as described with reference to FIGS. 1A-1B. Such hybridization may be performed in solution, e.g., by contacting bead 101-A in solution with polynucleotides S1 in solution. Alternatively, such hybridization may be performed using a bead 101-A that is already coupled to a substrate surface (e.g., within a flowcell and/or well) and then contacting that bead with polynucleotides S1 which is in solution.

Method 600 also may include adding first labeled nucleotides to the first polynucleotides using a sequence of the second polynucleotides (operation 602). For example, first labeled nucleotides 121 may be added to first polynucleotides C1 using a sequence of second polynucleotides S1 in a manner such as described with reference to FIG. 1C. It will be appreciated that first labeled nucleotides 121 need not necessarily be the same type of nucleotide as one another. For example, the first labeled nucleotides that are added may include a mixture of nucleotides corresponding to different SNPs that may be present in the second polynucleotides.

Method 600 also may include measuring a first signal intensity from the first labeled nucleotides (operation 603). For example, detection circuitry 160 may measure a first signal intensity from first labeled nucleotides 121 in a manner such as described with reference to FIG. 1C.

In some examples, method 600 also may include adding second labeled nucleotides to the first polynucleotides using the sequence of the second polynucleotides (operation 604). For example, second labeled nucleotides 131 may be added to first polynucleotides C1 using a sequence of second polynucleotides S1 in a manner such as described with reference to FIG. 1D.

Method 600 also may include measuring a second signal intensity from the second labeled nucleotides (operation 605). For example, detection circuitry 160 may measure a second signal intensity from second labeled nucleotides 131 in a manner such as described with reference to FIG. 1D.

Method 600 also may include normalizing the first signal intensity using the second signal intensity, the normalized first signal intensity characterizing the second polynucleotides in the sample (operation 606). For example, detection circuitry 160 may include or may be coupled to a processor coupled to a non-transitory computer-readable medium. The computer-readable medium may store instructions for causing the processor to normalize the first signal intensity from first labeled nucleotides 121, 122, or 123 using the second signal intensity from second labeled nucleotides 131, 132, or 133, respectively. Illustratively, the instructions may cause the processor to divide the first signal intensity from first labeled nucleotides 121, 122, or 123 by the second signal intensity from second labeled nucleotides 131, 132, or 133 in a manner such as described with reference to FIGS. 4A-4B. The instructions may cause the processor to output the normalized first signal intensity, e.g., to the same or different computer-readable medium, or a display screen coupled to the processor, or to another operation being performed by the processor. Illustratively, the instructions may cause the processor to identify the first labeled nucleotides (make a base call) using the normalized first signal intensity, e.g., by correlating the normalized first signal intensity to the base of the first labeled nucleotides. Additionally, or alternatively, the instructions may cause the processor to calculate an amount of the second polynucleotides in the sample, e.g., by correlating the normalized first signal intensity to an amount of second polynucleotides in the sample.

Note that labeled nucleotides 141 described with reference to FIG. 1E may be considered to be the “second” labeled nucleotides to which operation 604 refers. As a still further option, signal intensity from both labeled nucleotides 131 and labeled nucleotides 141 may be measured in separate instances of operations 604-605, and a decision made as to which of such signal intensities is used to perform the normalization of operation 606 in a manner such as described elsewhere herein.

It will be appreciated that process flows such as described with reference to FIG. 6 suitably may be used to correct for many different sources of signal intensity variations that are not directly related to the identity of the labeled nucleotides being added or to the amount of polynucleotides in the sample. For example, operations 601-0606 may be performed for beads 101-A, 101-B, 101C to obtain a normalized signal intensity for labeled nucleotides 121 that become coupled to beads of that type, and also may be performed (e.g., in parallel) for other beads of other types (that is, coupled to other capture oligonucleotides that are specific to other target polynucleotides or other analytes), to obtain a normalized signal intensity for those beads. The instructions may cause the processor to calculate differences or ratios between amounts of different polynucleotides in the sample using differences or ratios between the normalized signal intensity for beads that are selective to those polynucleotides. Such normalizing may, for example, correct for difference between the signal intensities for labeled nucleotides arising from any combination of effects such as described elsewhere herein.

Illustratively, such normalizing may correct for a difference in signal intensity that is caused by a bead position. Additionally, or alternatively, such normalizing may, for example, correct for a difference in signal intensity that is caused by a surface characteristic of a well. Additionally, or alternatively, such normalizing may, for example, correct for a difference in signal intensity that is caused by a loading condition of a bead. Additionally, or alternatively, such normalizing may, for example, correct for a difference in signal intensity that is caused by a capture efficiency of a bead to a substrate surface. Additionally, or alternatively, such normalizing may, for example, correct for a difference in signal intensity that is caused by a size of a bead. Additionally, or alternatively, such normalizing may, for example, correct for a difference in signal intensity that is caused by a number of target polynucleotides coupled to a bead.

Accordingly, the present normalization techniques may be used to characterize target polynucleotides or other analytes in a sample with significantly improved accuracy. It will be appreciated that the resulting characterization may be utilized in any suitable application, such as genotyping, NIPT, DNA methylation, or assays such as described in PCT Publication No. WO2021/074087. In one nonlimiting example, the normalized signal intensity for labeled nucleotides that are added corresponds to an amount of a particular single nucleotide polymorphism (SNP) in the sample. In another nonlimiting example, the normalized first signal intensity for labeled nucleotides that are added corresponds to an amount by which a particular base is methylated in the sample. Other examples readily may be envisioned.

### Working Examples

The following examples are intended to be purely illustrative, and not limiting.

Signal normalizations such as described with reference to FIGS. 1A-1E, 2A-2B, 3A-3B, 4A-4B, and 5 were performed using the HiSeq (unpatterned flowcell) and iSeq (patterned flowcell) sequencing platforms, both commercially available from Illumina, Inc. (San Diego, California). For these normalizations, five cycles of single base addition plus intensity reads (1^{st} cycle is SNP) were performed using captured whole genome amplification (WGA) DNA target on silica beads with complementary probe oligonucleotides.

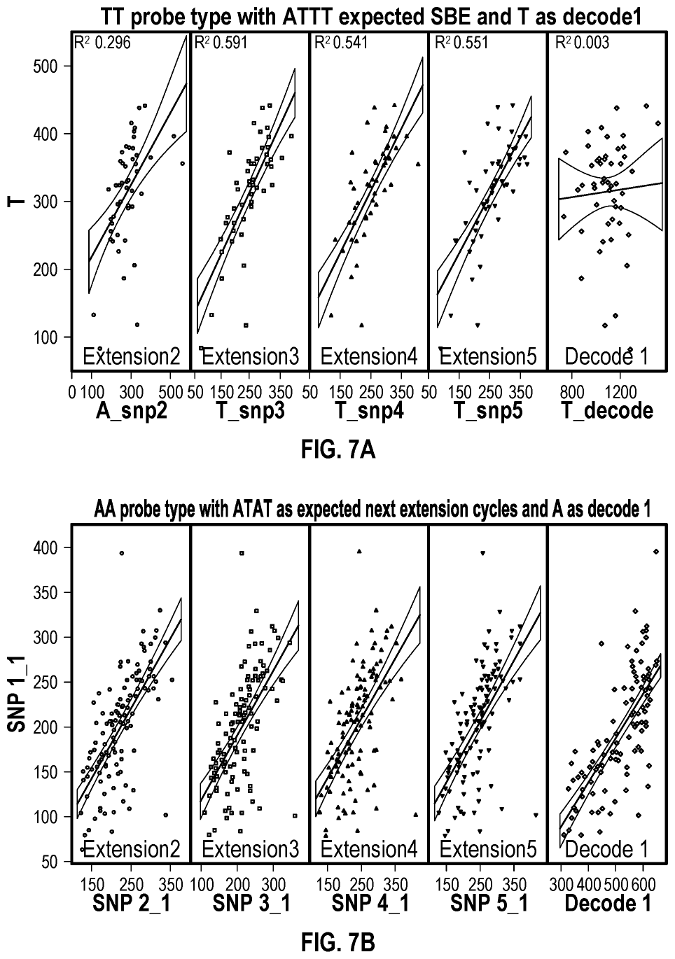

FIGS. 7A-7B illustrate example raw signal intensities that were obtained using compositions and operations similar to that described with reference to FIGS. 1C and 1D, and in which the beads were coupled to an unpatterned flowcell in the HiSeq and a patterned flowcell in the iSeq. In the nonlimiting example illustrated in FIG. 7A, a mixture of different bead types (beads coupled to different capture oligonucleotides) was used, and the target polynucleotide had sequence ATAAA for which the capture oligonucleotide was expected to be extended in subsequent cycles using the sequence TATTT of labeled nucleotides. The A, T, T, and T indicated in bold in that sequence respectively are referred to in FIG. 7A as “Extension2,” “Extension3,” “Extension4,” and “Extension5.” An additional labeled T nucleotide was used in another cycle to decode the barcode sequence, referred to as “Decode1” in FIG. 7A. Each of the data points shown in FIG. 7A corresponds to the raw total intensity obtained from a corresponding bead to which the labeled nucleotide T was coupled in the initial SNP cycle, as determined by the fluorescence wavelength. As shown in the shaded areas of FIG. 7A, a positive correlation was observed for extension cycle intensities with SNP cycle intensity. For example, a dim cycle for SNP was observed also to be dim for each of the extension cycles, but not with the decode cycle.

In the nonlimiting example illustrated in FIG. 7B, a mixture of different bead types was used, and the target polynucleotide had sequence TTATA for which the capture oligonucleotide was expected to be extended in subsequent cycles using the sequence AATAT of labeled nucleotides. The A, T, A, and T indicated in bold in that sequence respectively are referred to in FIG. 7B as “Extension2,” “Extension3,” “Extension4,” and “Extension5.” An additional labeled A nucleotide was used in another cycle to decode the barcode sequence, referred to as “Decode1” in FIG. 7B. Each of the data points shown in FIG. 7B corresponds to the raw total intensity obtained from a corresponding bead to which the labeled nucleotide A was coupled in the initial SNP cycle, as determined by the fluorescence wavelength. As shown in the shaded areas of FIG. 7B, a positive correlation was observed for extension cycle intensities, and for the decode cycle, with SNP cycle intensity. For example, a dim cycle for SNP was observed also to be dim for each of the extension cycles and for the decode cycle.

Accordingly, it may be understood from FIGS. 7A-7B that intensities from all extension cycles were positively correlated with SNP cycle intensity for both HiSeq (FIG. 7A) and iSeq (FIG. 7B), as expected. However, decode cycle and SNP cycle intensities were observed to be correlated for iSeq and not for HiSeq, and this difference was attributed to the patterned flowcell surface of iSeq. This was interpreted as confirming that signal variability due to target capture efficiency may be normalized using one or more extra cycles of probe extension, and that signal variability due to non-uniform bead loading on patterned flowcells may be normalized using decode cycles and/or one or more extra cycles of probe extension.

Because the extra cycle(s) of probe extension were observed to be correlated with assay cycle intensity, such correlation was used to normalize the assay signal from the beads. The normalization process used for the 4-dye HiSeq system is described below, and readily may be adapted for 1-dye systems.

1) After decoding, expected base incorporation for the extension cycles for all the beads are known. For each extension cycle, mean intensity for specific bases A/C/G/T were calculated for all the beads that have that specific base as the expected base call.

2) For each extension cycle, a normalization factor was calculated for each bead by dividing the mean intensity of the expected call/observed signal. For example, if a bead is expected to have A as base call for extension cycle 2, then the normalization factor was (mean of A channel signals for all the beads with expected A signal for extension2/observed A channel signal for the bead).

3) After repeating this process for extension cycles 2-5, four normalization factors for each bead were calculated. The average of these four factors was used for final normalization.

4) Intensities from all four channels for each bead were multiplied by the average normalization factor, and normalized intensities were obtained.

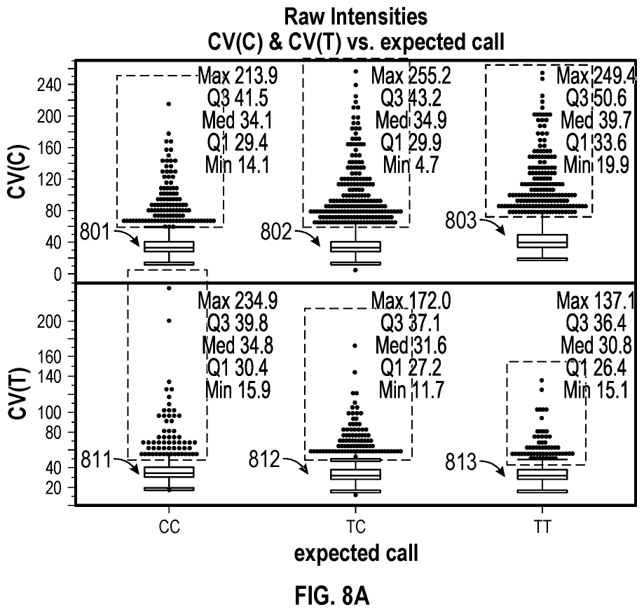

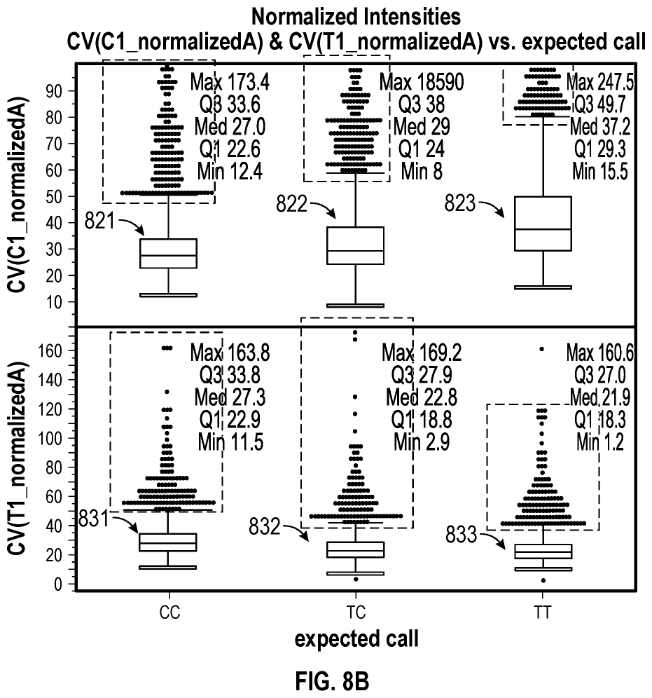

FIGS. 8A-8B respectively illustrate example mean coefficients of variation based on raw and normalized signal intensities obtained using a HiSeq instrument in a manner such as described with reference to FIGS. 1C-1D and 7A-7B. More specifically, a mixture of different bead types (beads coupled to different capture oligonucleotides) was used. Raw total intensities were obtained from beads to which either the labeled nucleotide T or the labeled nucleotide C was coupled in the initial SNP cycle, as determined by the fluorescence wavelengths. Error bars are shown about the different median CVs. The regions enclosed by the dashed lines were excluded from the analysis as they represent outliers detected by the algorithm used by the software (JMP) for creating box plot. More details of the feature can be found at https://www.jmp.com/support/help/en/16.0/index.shtml #page/jmp/outlier-box-plot.shtml #ww81387.

Plots 801-803 in FIG. 8A represent the mean value of coefficient of variation (CV) grouped with expected call, based on the raw signal intensities for the expected or unexpected labeled nucleotides (e.g. C intensity is expected for beads with pure C expected signal or mixture of TC signal. However, C intensity is unexpected for beads with pure T expected signal). Plot 801 represents the median CV for C channel intensity for all beads that were expected to have pure C signal (corresponding to condition 3 in FIG. 5), plot 802 represents the median CV for C channel intensity of all beads that were expected to have a mixture of T and C signal (corresponding to condition 2 in FIG. 5), and plot 803 represents the median CV for C channel intensity for all beads that were expected to have pure T signal (corresponding to condition 1 in FIG. 5). Illustratively, the median CV for plot 801 was 34.1%, the median CV for plot 802 was 34.9%, and the median CV for plot 803 was 39.7%. Plots 811-813 in FIG. 8A represent the median CV grouped with expected call, based on the raw signal intensities for expected or unexpected labeled nucleotides as described above. For example, plot 811 represents the median CV for T channel intensity for all beads that were expected to have pure C signal (corresponding to condition 3 in FIG. 5), plot 812 represents the median CV for T channel intensity of all beads that were expected to have a mixture of T and C signal (corresponding to condition 2 in FIG. 5), and plot 813 represents the median CV for T channel intensity for all beads that were expected to have pure T signal (corresponding to condition 1 in FIG. 5). Illustratively, the median CV for plot 811 was 34.8%, the median CV for plot 812 was 31.6%, and the median CV for plot 813 was 30.8%.

The raw signals were normalized in a manner using operations 1) to 4) described above. Plots 821-823 in FIG. 8B represent the mean CV grouped with expected call, based on the normalized signal intensities for the expected or unexpected labeled nucleotides in a manner similar to that described for the raw signals with reference to plots 801-803. The median CV for plot 821 was 27.3% (an improvement of about 6.8% as compared to the raw data), the median CV for plot 822 was 29% (an improvement of about 5.9% as compared to the raw data), and the median CV for plot 823 was 37.2% (an improvement of about 2.5% as compared to the raw data). Plots 831-833 in FIG. 8B represent the mean CV grouped with expected call, based on the normalized signal intensities for expected or unexpected labeled nucleotides in a manner similar to that described for the raw signals with reference to plots 811-813. Illustratively, the median CV for plot 831 was 27.3% (an improvement of about 7.5% as compared to the raw data), the median CV for plot 832 was 22.8% (an improvement of about 8.8% as compared to the raw data), and the median CV for plot 833 was 21.9% (an improvement of about 8.9% as compared to the raw data).

Accordingly, from FIGS. 8A-8B, it may be understood that for probes with T/C as the expected SNP type, the normalization process reduced the coefficient of variation (CV) within SNP intensities for each probe type by up to about 9% on the HiSeq (compare FIG. 8B to FIG. 8A). Accordingly, it may be understood that the normalization process increased the accuracy of SNP calls by about 2-9% based on the base call mechanism used.

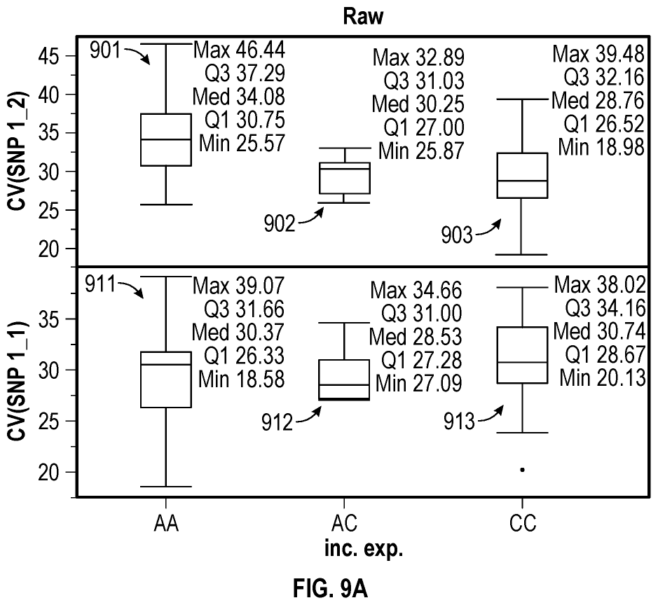

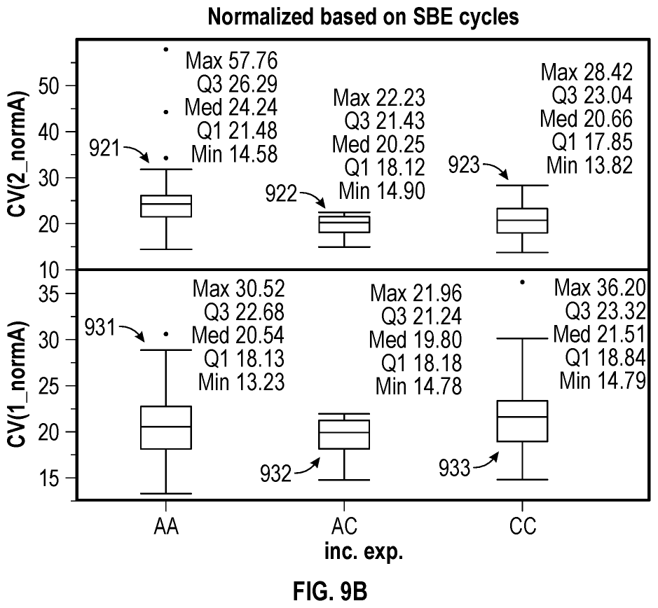

A similar normalization strategy was used for normalizing the signal for the iSeq (1-dye system). FIGS. 9A-9B respectively illustrate example median coefficients of variation based on raw and normalized signal intensities obtained using an iSeq instrument in a manner such as described with reference to FIGS. 1C-1D, 7A-7B, and 8A-8B. Error bars are shown about the different median CVs. Plots 901-903 in FIG. 9A were obtained in a similar manner as described for plots 801-803. Plot 901 represents the median CV for C channel intensity for all beads that were expected to have pure A signal (corresponding to condition 1 in FIG. 5), plot 902 represents the mean CV for C channel intensity of all beads that were expected to have a mixture of A and C signal (corresponding to condition 2 in FIG. 5), and plot 903 represents the median CV for C channel intensity for all beads that were expected to have pure C signal (corresponding to condition 3 in FIG. 5). Illustratively, the median CV for plot 901 was about 34.1%, the median CV for plot 902 was about 30.3%, and the median CV for plot 903 was about 28.8%. Plots 911-913 in FIG. 9A represent the median CV grouped with expected call, based on the raw signal intensities for expected or unexpected labeled nucleotides For example, plot 911 represents the median CV for A channel intensity for all beads that were expected to have pure A signal (corresponding to condition 1 in FIG. 5), plot 912 represents the median CV for A channel intensity of all beads that were expected to have a mixture of A and C signal (corresponding to condition 2 in FIG. 5), and plot 913 represents the median CV for A channel intensity for all beads that were expected to have pure C signal (corresponding to condition 3 in FIG. 5). Illustratively, the median CV for plot 911 was about 30.4%, the median CV for plot 912 was about 28.5%, and the median CV for plot 913 was about 30.7%.

The raw signals were normalized in a manner using operations 1) to 4) described above. Plots 921-923 in FIG. 9B represent the median CV grouped with expected call, based on the normalized signal intensities for the expected or unexpected labeled nucleotides in a manner similar to that described for the raw signals with reference to plots 901-903. The median CV for plot 921 was about 24.2% (an improvement of about 9.9% as compared to the raw data), the median CV for plot 922 was about 20.3% (an improvement of about 10.0% as compared to the raw data), and the median CV for plot 923 was about 20.7% (an improvement of about 8.1% as compared to the raw data). Plots 931-933 in FIG. 9B represent the median CV grouped with expected call, based on the normalized signal intensities for expected or unexpected labeled nucleotides in a manner similar to that described for the raw signals with reference to plots 911-913. Illustratively, the median CV for plot 931 was about 20.5% (an improvement of about 9.9% as compared to the raw data), the median CV for plot 932 was about 19.8% (an improvement of about 8.7% as compared to the raw data), and the median CV for plot 933 was about 21.5% (an improvement of about 9.2% as compared to the raw data).

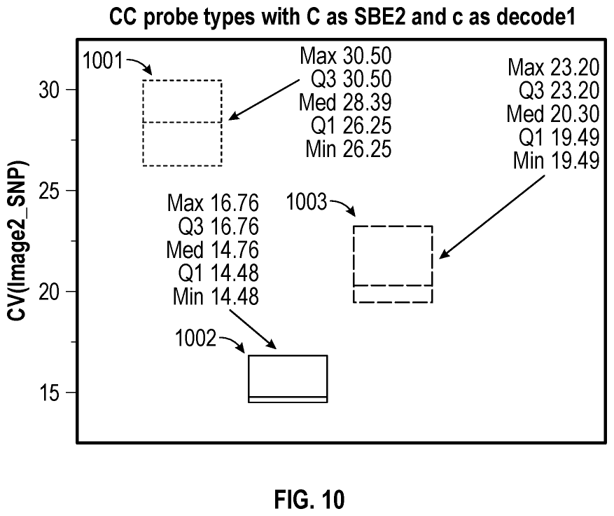

FIG. 10 illustrates example median coefficients of variation based on raw and normalized signal intensities obtained using an Illumina iSeq instrument in a manner such as described with reference to FIGS. 1C-1E, 7A-7B, 8A-8B, and 9A-9B. Error bars are shown about the different mean CVs. Plot 1001 in FIG. 10 corresponds to raw data for addition of C; plot 1002 corresponds to that raw data normalized using another extension cycle using operations 1) to 4) described above; and plot 1003 corresponds to that raw data normalized using a decode cycle using operations 1) to 4) described above. The median CV for plot 1001 was about 28.4%, the median CV for plot 1002 was about 14.8% (an improvement of about 13.6% as compared to the raw data), and the median CV for plot 1003 was about 20.3% (an improvement of about 8.1% as compared to the raw data).

From FIGS. 9A-9B, it may be understood that normalization by extra probe extension cycles decreased the CV by about 10% for SNP intensities (compare FIG. 9B to FIG. 9A) for A/C SNP type. From FIG. 10, it also may be understood that for all probes with expected probe type as CC and same expected base call C for extension cycle 2 and decode cycle 1 (decode1), CV decreased by about 14% through normalization using extension cycle 2 and about 8% through normalization using decode cycle 1 (FIG. 10).

Accordingly, it may be understood that the extra probe cycle extension-based normalization approach works well when signal from extra probe cycles correlates with assay cycle. It was observed that signal from all extension cycles is correlated with the first SNP cycle; accordingly, any extension cycle may be usable for normalizing the first SNP cycle, or any other extension cycle. To account for potential variability between correlation of a particular extension cycle intensity and SNP cycle intensity, an average normalization factor was used although other suitable calculations for normalization may be implemented such as described elsewhere herein.

### Additional Comments

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

While various illustrative examples are described above, it will be apparent to one skilled in the art that various changes and modifications may be made therein without departing from the invention. The appended claims are intended to cover all such changes and modifications that fall within the true spirit and scope of the invention.

What is claimed is:

1. A method for characterizing polynucleotides in a sample, the method comprising:
hybridizing first polynucleotides coupled to a first substrate to second polynucleotides in a sample;
adding first labeled nucleotides to the first polynucleotides using a sequence of the second polynucleotides;
measuring a first signal intensity from the first labeled nucleotides;
adding second labeled nucleotides to the first polynucleotides using the sequence of the second polynucleotides;
measuring a second signal intensity from the second labeled nucleotides; and
normalizing the first signal intensity using the second signal intensity, the normalized first signal intensity characterizing the second polynucleotides in the sample.

2. The method of claim 1, wherein the second labeled nucleotides are added after the first labeled nucleotides are added.

3. The method of claim 1, wherein the second labeled nucleotides are added before the first labeled nucleotides are added.

4. The method of any one of claims 1 to 3, further comprising:
hybridizing third polynucleotides coupled to a second substrate to fourth polynucleotides in the sample;
adding third labeled nucleotides to the third polynucleotides using a sequence of the fourth polynucleotides;
measuring a third signal intensity from the third labeled nucleotides;
adding fourth labeled nucleotides to the third polynucleotides using the sequence of the fourth polynucleotides;
measuring a fourth signal intensity from the fourth labeled nucleotides; and
normalizing the third signal intensity using the fourth signal intensity, the normalized third signal intensity characterizing the fourth polynucleotides in the sample.

5. The method of claim 4, further comprising calculating a difference or ratio between an amount of the second polynucleotides in the sample and an amount of the fourth polynucleotides in the sample using a difference or ratio between the normalized first signal intensity and the normalized third signal intensity.

6. The method of claim 4 or claim 5, wherein the fourth polynucleotides have a different sequence than the second polynucleotides.

7. The method of any one of claims 4 to 6, wherein the first substrate comprises a first bead, and wherein the second substrate comprises a second bead.

8. The method of claim 7, wherein the first bead is located within a first well, and the second bead is located within a second well.

9. The method of claim 8, wherein the normalizing corrects for a difference between the first signal intensity and the third signal intensity that is caused by a position of the first bead within the first well or a position of the second bead within the second well.

10. The method of claim 8 or claim 9, wherein the normalizing corrects for a difference between the first signal intensity and the third signal intensity that is caused by surface characteristic of the first well or a surface characteristic of the second well.

11. The method of any one of claims 8 to 10, wherein the normalizing corrects for a difference between the first signal intensity and the third signal intensity that is caused by a loading condition of the first bead within the first well or a loading condition of the second bead within the second well.

12. The method of any one of claims 8 to 11, wherein the normalizing corrects for a difference between the first signal intensity and the third signal intensity that is caused by a capture efficiency of the first bead within the first well or a capture efficiency of the second bead within the second well.

13. The method of any one of claims 7 to 12, wherein the normalizing corrects for a difference between the first signal intensity and the third signal intensity that is caused by a size of the first bead or a size of the second bead.

14. The method of any one of claims 4 to 13, wherein the normalizing corrects for a difference between the first signal intensity and the third signal intensity that is caused by a number of the second polynucleotides coupled to the first substrate or a number of the fourth polynucleotides coupled to the second substrate.

15. The method of any one of claims 4 to 14, wherein the normalized first signal intensity corresponds to an amount of a first single nucleotide polymorphism (SNP) in the sample, and the normalized third signal intensity corresponds to an amount of a second, different SNP in the sample.

16. The method of any one of claims 4 to 14, wherein the normalized first signal intensity corresponds to an amount by which a first base is methylated in the sample, and the normalized third signal intensity corresponds to an amount by which the first base is not methylated in the sample.

17. The method of any one of claims 1 to 16, wherein the first and second polynucleotides are hybridized to one another in solution.

18. The method of any one of claims 1 to 17, wherein the first substrate comprises a bead.

19. The method of claim 18, further comprising capturing the bead within a well.

20. The method of claim 19, wherein the first and second labeled nucleotides are added after the bead is captured in the well.

21. The method of claim 19 or claim 20, wherein the first and second signal intensities are measured using a complementary metal oxide semiconductor (CMOS) sensor on which the well is disposed.

22. The method of any one of claims 1 to 21, wherein the second labeled nucleotide is added to a position adjacent to where the first labeled nucleotide is added.

23. The method of any one of claims 1 to 22, further comprising adding one or more additional nucleotides to positions between where the first and second labeled nucleotides respectively are added.

24. The method of any one of claims 1 to 23, wherein the first and second labeled nucleotides respectively comprise first and second fluorophores.

25. The method of any one of claims 1 to 24, wherein characterizing the second polynucleotides in the sample comprises determining an amount of the second polynucleotides in the sample, identifying the first nucleotide, or both determining an amount of the second polynucleotides in the sample and identifying the first nucleotide.

26. The method of any one of claims 1 to 25, wherein the second labeled nucleotide is coupled to a primer hybridized to a barcode oligonucleotide coupled to the first substrate.
